(19) United States
(12) Patent Application Publication
(10) Pub. No.: US 2004/0087635 A1
(43) Pub. Date: May 6, 2004

(54) Novel treatment

(76) Inventor: Jan Hammarsten, Varberg (SE)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/433,037

(22) PCT Filed: Nov. 29, 2001
(86) PCT No.: PCT/EP01/13950

(30) Foreign Application Priority Data
Nov. 29, 2000 (GB) 0029125.2

(51) Int. Cl. A61K 31/426; A61K 31/4439

(52) U.S. Cl. 514/369; 514/342

(57) ABSTRACT

A method for delaying or preventing an increase in prostate gland volume in humans or non-human mammals, which method comprises the administration of an effective, non-toxic and pharmaceutically acceptable amount of an insulin sensitiser or a pharmaceutically acceptable derivative thereof.

This invention relates to a novel treatment and in particular to a method for the treatment and/or prophylaxis of benign prostatic hyperplasia or clinical prostate cancer.

European Patent Application, Publication Number 0306228 discloses certain thiazolidinedione derivatives which are disclosed inter alia as having hypoglycaemic and hypolipidaemic activity and activity in treating certain eating disorders. The compound of example 30 of EP 0306228 is 5-(4-[2-(N-methyl-N-(2-pyridyl)amino)ethoxy]benzyl)-2,4-thiazolidinedione (or ‘Compound (I)’).

Compound (1) is an example of a class of an anti-hyperglycaemic agent known as an ‘insulin sensitiser’. In particular Compound (I) is a thiazolidinedione insulin sensitiser.

European Patent Applications, Publication Numbers: 0306228, 0008203, 0139421, 0032128, 0428312, 0489663, 0155845, 0257781, 0208420, 0177353, 0319189, 0332331, 0332332, 0528734, 0508740; International Patent Application, Publication Numbers 92/18501, 93/02079, 93/22445 and U.S. Pat. Nos. 5,104,888 and 5,478,852, also disclose certain thiazolidinedione derivatives which are stated to have hypoglycaemic and hypolipidaemic activity.

Another series of compounds generally recognised as having insulin sensitiser activity are those typified by the compounds disclosed in International Patent Applications, Publication Numbers WO93/21166 and WO94/01420. These compounds are herein referred to as ‘acyclic insulin sensitisers’. Other examples of acyclic insulin sensitisers are those disclosed in U.S. Pat. No. 5,232,945 and International Patent Applications, Publication Numbers WO92/03425 and WO91/19702.

Examples of other insulin sensitisers are those disclosed in European Patent Application, Publication Number 0533933, Japanese Patent Application Publication Number 05271204 and U.S. Pat. No. 5,264,451.

Non-thiazolidinedione insulin sensitisers include the compounds of formula (I) of International application, publication number WO 97/31907.

The above mentioned publications are incorporated herein by reference.

The prostate is a walnut-sized gland located just below the bladder and surrounding part of the urethra Enlargement of the prostate or benign prostatic hyperplasia (hereinafter also referred to as “BPH”) is the most common benign neoplasm (non-cancerous enlargement of the prostate gland) in men and has a high prevalence that increases with age. Enlargement of the prostate in BPH occurs primarily in an area known as the transition zone (hereinafter also referred to as “TZ”).

Although BPH can develop without any serious symptoms, common complaints include frequent urination, decreased urine flow and nocturia. Severe BPH can result in serious problems over time. Urine retention and strain on the bladder can lead to urinary tract infections, bladder or kidney damage, bladder stones, and incontinence. Current treatment options for relief of symptoms include treatment with _{α}-1-adrenergic receptor blockers that inhibit contraction of prostatic smooth muscle, 5-_{α}-reductase inhibitors and surgery.

The cause and contributing factor or factors to onset and progression of BPH are not fully understood. Recently it has been suggested that non-insulin dependent diabetes (NIDDM), treated hypertension, tallness, obesity, low HDL cholesterol levels and high insulin levels constitute risk factors for the development of BPH (Prostate Cancer and Prostatic Diseases 1998, 1, 157-162, Hammarsten et al. Blood Pressure 1999, 8, 29-36, Hammarsten et al and E Urology, 2001, 39, 151, Hammarsten et al and Submitted to publication, Hammarsten et al)

Prostate cancer is a malignant tumour growth within the prostate gland. Currently the cause of prostate cancer is unknown. Recently, it has been suggested that a large prostate gland volume, fast BPH-growth rate, treated hypertension, obesity, dyslipidaemia and hyperinsulinaemia constitute risk factors for the development of clinical prostate cancer as measured by stage and grade (Hammarsten et al. In preparation). Clinical prostate cancer is currently one of the most prevalent cancers in men.

It is now indicated that hyperinsulinaemia is the primary causal factor in the onset and development of BPH. It is therefore considered that ameliorating the hyperinsulinaemia by treatment with an insulin sensitiser, such as Compound (I), will have a significant beneficial effect upon both the onset and progression of BPH.

It is further indicated that the development of clinical prostate cancer is linked to treated hypertension, obesity, dyslipidaemia and to high plasma insulin levels and that moreover the total prostate gland volume and the rate of development of BPH is positively correlated with the onset and development of clinical prostate cancer as measured by stage and grade (Hammarsten et al. In preparation). Thus treatment of high risk individuals (those having treated hypertension, obesity, dyslipidaemia, hyperinsulinaemia, large prostate gland volume or fast growing BPH) with an agent that improves insulin sensitivity would be expected to inhibit or prevent the onset or development of clinical prostate cancer.

Finally, it is also indicated the onset of BPH and its progression can be monitored by measurement of changes in total prostate gland volume rather than the much more complex measurement of change in TZ volume.

Accordingly, in a first aspect, the invention provides a method for delaying or preventing an increase in prostate gland volume, suitably total prostate gland volume, in humans or non-human mammals, which method comprises the administration of an effective, non-toxic and pharmaceutically acceptable amount of an insulin sensitiser, such as Compound (I), or a pharmaceutically acceptable derivative thereof.

Suitably, the increase in total prostate gland volume is associated with the onset and/or development of benign prostatic hyperplasia (BPH).

Preferably the treatment delays an increase in prostate gland volume.

Preferably the treatment delays or prevents an increase in volume of the transition zone (TZ) of the prostate gland.

Of particular interest is the delay or prevention of prostate volume increase in fast growing BPH and also in men with associated risk factors It is of most especial interest that the delay or prevention of increase in prostate gland volume results in a delay or prevention of onset of clinical prostate cancer, particularly in those having fast growing BPH.

In a further aspect there is provided a method for the treatment and/or prophylaxis of benign prostatic hyperplasia, in humans or non-human mammals, which method comprises the administration of an effective, non-toxic and pharmaceutically acceptable amount of an insulin sensitiser, such as Compound (1), or a pharmaceutically acceptable derivative thereof.

In yet a further aspect the invention provides a method for delaying or preventing the onset the of clinical p5rostate cancer, such as that resulting from BPH especially fast growing BPH, in humans or non-human mammals, which method comprises the administration of an effective, non-toxic and pharmaceutically acceptable amount of an insulin sensitiser, such as Compound (I), or a pharmaceutically acceptable derivative thereof.

Suitable insulin sensitisers include thiazolidinediones, especially thiazolidine-2,4-diones, that is a compound comprising a moiety of formula (A):

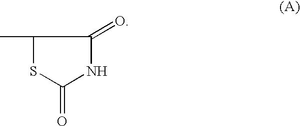

Suitable compounds comprising a moiety of formula (a) include compounds of formula (I):

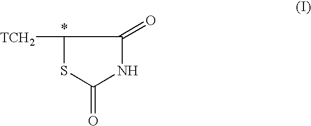

or a tautomeric form thereof and/or a pharmaceutically acceptable salt thereof and/or a pharmaceutically acceptable solvate thereof, wherein T represents an aryl or heterocyclyl group optionally substituted with one or more alkyl groups, aralkyl groups or heterocyclylalkyl groups, the said alkyl, aralkyl and heterocyclylalkyl groups themselves being optionally substituted.

Suitably, the carbon atom marked with an asterisk (*) in formula (1) is a chiral carbon.

In particular T represents a moiety selected from the list consisting of (a), (b), (c), (d), (e), (f), (g), (h) and (i):

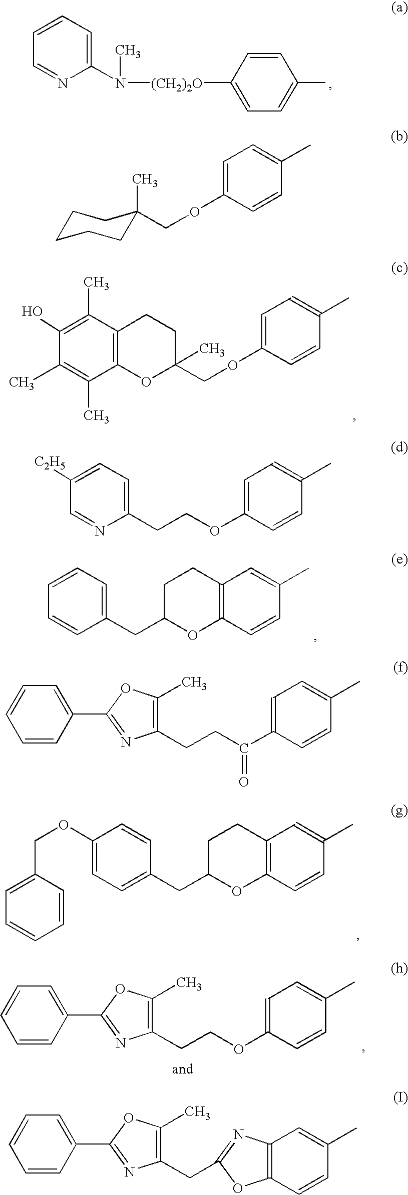

In particular should be mentioned the moieties of formula (a), (b), (c), (d) and (e).

Also included in the treatment of the invention are the insulin sensitisers disclosed in European Patent Applications, Publication Numbers: 0306228, 0008203, 0139421, 0032128, 0428312, 0489663, 0155845, 0257781, 0208420, 0177353, 0319189, 0332331, 0332332, 0528734 and 0508740, International Patent Application, Publication Numbers 92/18501, 93/02079, 93/22445 and U.S. Pat. Nos. 5,104,888 and 5,478,852, especially the specific example thereof. The contents of these publications are included herein by reference.

Thiazolidinedione insulin sensitisers may exist in one of several tautomeric forms, all of which are encompassed by the present invention as individual tautomeric forms or as mixtures thereof. Where an insulin sensitiser contains a chiral carbon, and hence exists in one or more stereoisomeric forms or where one or more geometric isomers exist, it will be appreciated that the method of the present invention encompasses all of the said forms of the insulin sensitiser whether as individual isomers or as mixtures of isomers, including racemates.

Particular examples of thiazolidinediones are those disclosed in EP 0306228 and WO94/05659. Further particular examples are the thiazolidinediones disclosed in EP0139421 and U.S. Pat. No. 5,478,852.

A preferred thiazolidinedione is Compound (I).

Further particular thiazolidinediones are, (+)-5-[[4-[(3,4-dihydro-6-hydroxy-2,5,7,8-tetramethyl-2H-1-benzopyran-2-yl)methoxy]phenyl]methyl]-2,4-thiazolidinedione (or troglitazone), 5-[4-[(1-methylcyclohexyl)methoxy]benzyl]thiazolidine-2,4-dione (or ciglitazone), 5-[4-[2-(5-ethylpyridin-2-yl)ethoxy]benzyl]thiazolidine-2,4-dione (or pioglitazone) or 5-[(2-benzyl-2,3-dihydrobenzopyran)-5-ylmethyl)thiazolidine-2,4-dione (or englitazone).

Suitable insulin sensitisers also include non-thiazolidinedione insulin sensitisers.

Suitable non-thiazolidinedione insulin sensitisers include the compounds of formula (1) of International application, publication number WO 97/31907 or a pharmaceutically acceptable derivative thereof. A particular compound of WO 97/31907 (or EP0888317) is 2(S)-(2-benzoyl-phenylamino)-3-{4-[2-5-methyl-2-phenyl-oxazol-4-yl)-ethoxy]-phenyl}-propionic acid or a pharmaceutically acceptable derivative thereof, such as a pharmaceutically acceptable salt or pharmaceutically acceptable solvate thereof.

The contents of WO 97/31907 (or EP0888317) are included herein by reference.

Suitable non-thiazolidinedione insulin sensitisers include the insulin sensitiser compounds specifically mentioned herein.

As used herein the term ‘clinical prostate cancer’ refers to a prostate cancer that threatens the life or well being of the host within his remaining normal life expectancy (Cancer: Principles and Practice of Oncology, Ed, Vincent T De Vita, Jr Samuel Hellman, Steven A Rosenberg, 4^{th} Ed. 1993, p1076).

As used herein ‘risk factors associated with prostate cancer’ in addition to BPH, especially fast growing BPH, include factors associated with high insulin levels such as NIDDM, treated hypertension, tallness, obesity and low HDL cholesterol levels.

When used herein the term ‘aryl’ includes phenyl and naphthyl optionally substituted with up to five, preferably up to three, groups selected from halogen, alkyl, phenyl, alkoxy, haloalkyl, hydroxy, amino, nitro, carboxy, alkoxycarbonyl, alkoxycarbonylalkyl, alkylcarbonyloxy, or alcylcarbonyl groups.

Suitable heterocyclyl groups are aromatic and non-aromatic heterocylic groups.

Suitable non-aromatic heterocylic groups include groups comprising single or fused ring heterocyclic groups comprising up to 4 hetero atoms in each ring selected from oxygen, sulphur or nitrogen, optionally fused to one or more aryl groups.

Suitable aromatic heterocyclyl groups include substituted or unsubstituted, single or fused ring aromatic heterocyclyl groups comprising up to 4 hetero atoms in each ring selected from oxygen, sulphur or nitrogen.

Favoured aromatic heterocyclyl groups include substituted or unsubstituted single ring aromatic heterocyclyl groups having 5 to 7 ring atoms, preferably 5 or 6 ring atoms.

In particular, the aromatic heterocyclyl groups comprise 1, 2 or 3 heteroatoms, especially 1 or 2, selected from oxygen, sulphur or nitrogen.

Suitable substituents for the heterocyclyl include up to 4 substituents selected from the group consisting of: alkyl, alkoxy, aryl and halogen or any two substituents on adjacent carbon atoms, together with the carbon atoms to which they are attached, may form an aryl group, preferably a benzene ring, and wherein the carbon atoms of the aryl group represented by the said two substituents may themselves be substituted or unsubstituted.

It will be appreciated that where the above-mentioned definitions of ‘aryl’, ‘heterocyclyl’ and the substituents thereof differ from those in the above mentioned patent publications with respect to the particular compounds disclosed therein, that the definitions in the said publications prevail.

When used herein the term ‘halogen’ refers to fluorine, chlorine, bromine and iodine; preferably chlorine.

When used herein the terms ‘alkyl’ and ‘alkoxy’ relate to groups having straight or branched carbon chains, containing up to 12 carbon atoms.

When used herein the term ‘acyl’ includes alkylcarbonyl groups.

Suitable alkyl groups are C₁₋₁₂ alkyl groups, especially C₁₋₆ alkyl groups e.g. methyl, ethyl, n-propyl, iso-propyl, n-butyl, isobutyl or tert-butyl groups.

Suitable substituents for any alkyl group include those indicated above in relation to the term “aryl”.

Suitable derivatives of insulin sensitisers are pharmaceutically acceptable derivatives, for example salts and solvates.

Suitable derivatives of any particular insulin sensitiser include those disclosed in the above mentioned publications.

Suitable pharmaceutically acceptable salts include salts derived from appropriate acids, such as acid addition salts, or bases.

Suitable pharmaceutically acceptable salts include metal salts, such as for example aluminium, alkali metal salts such as lithium, sodium or potassium, alkaline earth metal salts such as calcium or magnesium and ammonium or substituted ammonium salts, for example those with lower alkylamines such as triethylamine, hydroxy alkylamines such as 2-hydroxyethylamine, bis-(2-hydroxyethyl)-amine or tri-(2-hydroxyethyl)-amine, cycloalkylamines such as bicyclohexylamine, or with procaine, dibenzylpiperidine, N-benzyl-b-phenethylamine, dehydroabietylamine, N,N′-bisdehydroabietylamine, glucamine, N-methylglucamine or bases of the pyridine type such as pyridine, collidine, quinine or quinoline.

Suitable acid addition salts include pharmaceutically acceptable inorganic salts such as the sulphate, nitrate, phosphate, borate, hydrochloride and hydrobromide and pharmaceutically acceptable organic acid addition salts such as acetate, tartrate, maleate, citrate, succinate, benzoate, ascorbate, methane-sulphonate, a-keto glutarate and a-glycerophosphate, especially the maleate salt.

Suitable pharmaceutically acceptable salts of Compound (I) are as disclosed in EP 0306228 and WO94/05659 and include maleate salts.

Suitable pharmaceutically acceptable solvates include hydrates.

Suitable pharmaceutically acceptable solvates of Compound (I) are as disclosed in EP 0306228 and WO94/05659 and include hydrates.

As used herein the term ‘pharmaceutically acceptable’ embraces compounds, compositions and ingredients for both human and veterinary use: for example the term ‘pharmaceutically acceptable salt’ embraces a veterinarily acceptable salt.

The insulin sensitisers such as the thiazolidinediones, referred to herein are conveniently prepared according to the methods disclosed in the above mentioned patent publications in which they are disclosed: Thus, Compound (I), or the tautomeric form thereof, and/or a pharmaceutically acceptable salt thereof, and/or a pharmaceutically acceptable solvate thereof, may be prepared using the processes described in EP 0306228 and WO94/05659.

The salts and/or solvates of the thiazolidinediones may be prepared and isolated according to conventional procedures for example those disclosed in the, above mentioned, patent publications.

The present invention also provides an insulin sensitiser or a pharmaceutically acceptable derivative thereof, for use in the treatment and/or prophylaxis of BPH, especially in those with fast growing BPH or men with associated risk factors.

The present invention also provides an insulin sensitiser or a pharmaceutically acceptable derivative thereof, for use in the manufacture of a medicament for the treatment and/or prophylaxis of BPH, especially in those with fast growing BPH or men with associated risk factors.

In addition, there is provided an insulin sensitiser or a pharmaceutically acceptable derivative thereof, for use in delaying or preventing an increase in prostate gland volume, suitably total prostate gland volume, especially in those with fast growing BPH or men with associated risk factors.

Also provided is an insulin sensitiser or a pharmaceutically acceptable derivative thereof, for use in delaying or preventing the onset of clinical prostate cancer, such as that associated with, in particular, BPH especially fast growing BPH, or other risk factors associated with prostate cancer.

The invention further provides an insulin sensitiser or a pharmaceutically acceptable derivative thereof, for use in the manufacture of a medicament for delaying or preventing an increase in prostate gland volume, suitably total prostate gland volume or for delaying or preventing the onset the of clinical prostate cancer, such as that associated with, in particular, BPH especially fast growing BPH, or other risk factors associated with prostate cancer.

In the above-mentioned method the insulin sensitiser, may be administered per se or, preferably, as a pharmaceutical composition also comprising a pharmaceutically acceptable carrier.

In the treatment of the invention, the insulin sensitisers mentioned herein is formulated and administered in accordance with the methods disclosed in the above mentioned patent applications and patents.

Accordingly, the present invention also provides a pharmaceutical composition for the treatment and/or prophylaxis of benign prostatic hyperplasia, which composition comprises an insulin sensitiser, or a pharmaceutically acceptable derivative thereof, and a pharmaceutically acceptable carrier therefor.

The composition may, if desired, be in the form of a pack accompanied by written or printed instructions for use.

Usually the pharmaceutical compositions of the present invention will be adapted for oral administration, although compositions for administration by other routes, such as by injection and percutaneous absorption are also envisaged.

Particularly suitable compositions for oral administration are unit dosage forms such as tablets and capsules. Other fixed unit dosage forms, such as powders presented in sachets, may also be used.

In accordance with conventional pharmaceutical practice the carrier may comprise a diluent, filler, disintegrant, wetting agent, lubricant, colourant, flavourant or other conventional adjuvant.

Typical carriers include, for example, microcrystalline cellulose, starch, sodium starch glycollate, polyvinylpyrrolidone, polyvinylpolypyrrolidone, magnesium stearate, sodium lauryl sulphate or sucrose.

Suitable dosages of the insulin sensitiser include the known doses for these compounds as described in, or referred to, in reference texts such as the British and US Pharmacopoeias, Remington's Pharmaceutical Sciences (Mack Publishing Co.), Martindale, The Complete Drug Reference (London, The Pharmaceutical Press, 32nd Edition) or the above mentioned publications or doses which can be determined by standard procedures.

Suitable dosages of the Compound (1) include those disclosed in EP 0306228 and WO94/05659.

Suitable dosages of the Compound (1) include 1, 2, 3, 4, 5, 6, 7, 8, 9, 10, 11 or 12 mg of Compound (1), such as 2 to 4, 4 to 8 and 8 to 12 mg.

Particular dosages of Compound (1) are 2 mg, 4 mg and 8 mg.

Suitable doses of 2(S)-(2-benzoyl-phenylamino)-3-{4-[2-5-methyl-2-phenyl-oxazol-4-yl)-ethoxy]-phenyl}-propionic acid or a pharmaceutically acceptable derivative thereof, such as a pharmaceutically acceptable salt or pharmaceutically acceptable solvate thereof, are as described in WO 97/31907 (or EP0888317).

The composition of the invention may be administered from 1 to 6 times a day, but most preferably 1 or 2 times per day.

The solid oral compositions may be prepared by conventional methods of blending, filling or tabletting. Repeated blending operations may be used to distribute the active agent throughout those compositions employing large quantities of fillers. Such operations are of course conventional in the art. The tablets may be coated according to methods well known in normal pharmaceutical practice, in particular with an enteric coating.

Oral liquid preparations may be in the form of, for example, emulsions, syrups, or elixirs, or may be presented as a dry product for reconstitution with water or other suitable vehicle before use. Such liquid preparations may contain conventional additives such as suspending agents, for example sorbitol, syrup, methyl cellulose, gelatin, hydroxyethylcellulose, carboxymethylcellulose, aluminium stearate gel, hydrogenated edible fats; emulsifying agents, for example lecithin, sorbitan monooleate, or acacia; non-aqueous vehicles (which may include edible oils), for example almond oil, fractionated coconut oil, oily esters such as esters of glycerine, propylene glycol, or ethyl alcohol; preservatives, for example methyl or propyl p-hydroxybenzoate or sorbic acid; and if desired conventional flavouring or colouring agents.

For parenteral administration, fluid unit dosage forms are prepared utilizing the compound and a sterile vehicle, and, depending on the concentration used, can be either suspended or dissolved in the vehicle. In preparing solutions the compound can be dissolved in water for injection and filter sterilized before filling into a suitable vial or ampoule and sealing. Advantageously, adjuvants such as a local anaesthetic, a preservative and buffering agents can be dissolved in the vehicle. To enhance the stability, the composition can be frozen after filling into the vial and the water removed under vacuum. Parenteral suspensions are prepared in substantially the same manner, except that the compound is suspended in the vehicle instead of being dissolved, and sterilization cannot be accomplished by filtration. The compound can be sterilized by exposure to ethylene oxide before suspending in the sterile vehicle. Advantageously, a surfactant or wetting agent is included in the composition to facilitate uniform distribution of the compound.

Compositions may contain from 0.1% to 99% by weight, preferably from 10-60% by weight, of the active material, depending upon the method of administration. Compositions may, if desired, be in the form of a pack accompanied by written or printed instructions for use.

The compositions are formulated according to conventional methods, such as those disclosed in the publications mentioned above or in standard reference texts, for example the British and US Pharmacopoeias, Remington's Pharmaceutical Sciences (Mack Publishing Co.), Martindale, The Complete Drug Reference (London, The Pharmaceutical Press, 32nd Edition) and Harry's Cosmeticology (Leonard Hill Books).

No adverse toxicological effects are expected for the compositions or methods of the invention in the above mentioned dosage ranges.

TEST METHODS: Various methods can be used to examine the prostate gland, monitor increases in prostate gland size and hence the development and diagnosis of BPH, including ultrasound, digital rectal exam, urine flow testing, intravenous pyelogram (IVP), cystoscopy and prostate specific antigen (PSA).

Preferred methods for examining the prostate include digital rectal examination and ultrasound equipment (eg B & K Medical 3535).

A preferred method for monitoring prostate gland growth involves measurement of prostate gland volume change, for example by use of ultrasound (US) methods (see Liftrupp et al, Determination of Prostate Volume with Transrectal US for Cancer Screening; Part II; Accuracy of in-vitro and in-vivo techniques. Radiology 1991, 179, 49-53 and Terris M K et al, Determination of Prostate Volume by Transrectal Ultrasound, 1991, 45, 984-987.). Indeed, it is one component of the present invention that there is provided a method for monitoring the onset of BPH and its progression by measurement of changes in total prostate gland volume, using for example ultrasound (US) methodology

Clinical prostate cancer, when suspected, can be confirmed by use of conventional histopathological examination of for example ultrasound-guided core biopsies.

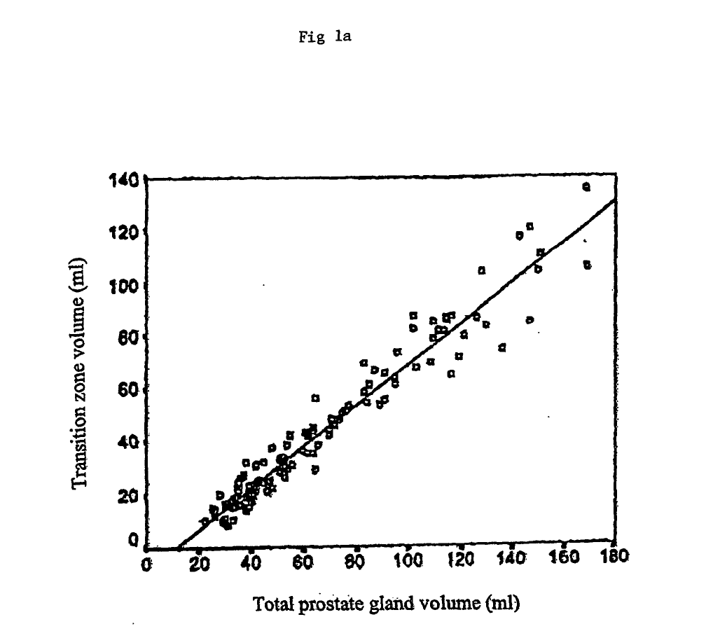

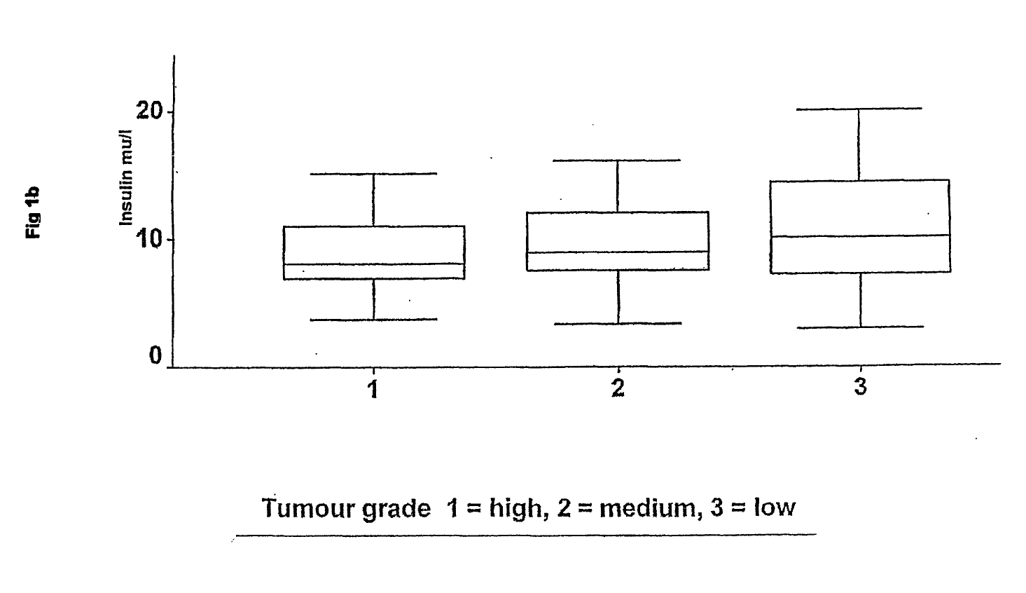

FIG. 1A shows the relationship between total prostate gland volume and tranzition zone volume (rs=0.97, n=114, p<0.0001).

FIG. 1B shows the relationship between clinical prostate cancer grade and fasting plasma insulin levels (mU/l).

The following discussion and data illustrate the invention but do not limit it in any way.

Pharmacological Data

Study 1: To Determine the Validity Coefficient of the Total Prostate Gland Volume as an Expression OF THE Transition Zone (TZ) Volume. To Test the Hypothesis OF Hyperinsulinaemia as a Causal Factor for the Development of Benign Prostatic Hyperplasia (BPH).

Summary

Patients and methods:—Three hundred and seven consecutive patients with lower urinary tract symptoms were studied. A subgroup of 114 patients were tested with regard to the validity coefficient between the total prostate gland volume and the TZ volume. In the total material of 307 men, a BPH risk factor analysis was performed in which groups of men with the following conditions were related to the annual BPH growth rate: men without or with metabolic disease, men with different components of the metabolic syndrome and men with low or high fasting plasma insulin values. The prostate gland volume and the TZ volume were determined using ultrasound. The presence of non-insulin-dependent diabetes mellitus (NIDDM) and treated hypertension was obtained from the patients' medical records. Data on blood pressure, waist and hip measurement, body height and weight were collected and body mass index (BMI) and waist/hip ratio (WHR) were calculated. Blood samples were drawn from fasting patients to determine the insulin and HDL-cholesterol values.

Results

In the subgroup of men subjected to measurement of both the total prostate gland volume and the TZ volume, the correlation coefficient between total prostate gland volume and the TZ volume was rs=0.97; p<0.0001 which, thus, constituted the validity coefficient. The median annual BPH growth rate in the total group was 1.03 ml/year. The median annual BPH growth rate was faster in men with metabolic disease (p<0.0001), NIDDM (p<0.0001), treated hypertension (p<0.0001), obesity (p<0.0001) and dyslipidaemia (p<0.0001) than in men without metabolic disease. Moreover, the annual BPH growth rate correlated positively with the diastolic blood pressure (rs=0.27; p<0.001), the BMI (rs=0.22; p<0.001) and four other expressions of obesity and negatively with the HDL-cholesterol level (rs=−0.15; p<0.001). The median annual BPH growth rate was faster in men with a high than in men with a low fasting plasma insulin level (p=0.019). When the patients were divided into quartiles, the median annual BPH growth rate increased statistically significantly with increasing fasting plasma insulin levels. The fasting plasma insulin values correlated with the annual BPH growth rates (p=0.009). When performing a multivariate analysis using the total prostate gland volume as dependent variable, fasting plasma insulin (p=0.001) and age (p<0.001) became statistically significant.

Conclusion

The results of the present report suggest that the total prostate gland volume constitutes a valid expression of BPH. The findings support the hypothesis that hyperinsulinaemia is causally related to the development of BPH and generate a hypothesis of an increased sympathetic nerve activity in men with BPH.

Introduction

Benign prostatic hyperplasia (BPH) is the most common benign disease in men older than 40 years of age. BPH is an enlargement of the prostate gland resulting from proliferation of stromal and glandular elements (1A-4A). The prevalence of BPH increases rapidly from 8% in the age interval between 31-40 and reaches 82% in the interval between 71-80 according to a compilation of autopsies involving 1075 men in four countries (5A). The majority of men with BPH have urinary symptoms requiring either surgery or medical therapy at a high cost for society (6A). Unfortunately, as long as the aetiology of BPH remains ambiguous, no long-term rational medical strategy for its treatment has been possible to establish.

Over the years, the most accepted hypothesis concerning the aetiology of BPH has been aberrations of the steroid hormones testosterone and oestrogen. Up to now, however, few data have linked aberrations of these hormones to the development of BPH (7A-14A).

We have recently proposed a hypothesis that hyperinsulinaemia is causally related to the development of BPH based on a clinical observation and two clinical studies in which the prostate gland volume was related to components of the metabolic syndrome (15A-16A). The studies gave evidence that non-insulin-dependent diabetes mellitus (NIDDM), hypertension, tallness, obesity, dyslipidaemia and hyperinsulinaemia were risk factors for the development of BPH. The results of these studies also suggest that BPH is a component of the metabolic syndrome and that BPH patients may share the same metabolic abnormality of a defective insulin-mediated glucose uptake and a secondary hyperinsulinaemia as patients with the metabolic syndrome. Thus, our studies supported the hypothesis that hyperinsulinaemia was causally related to the development of BPH and gave rise to another hypothesis of an increased sympathetic nerve activity in men with BPH.

In these previous studies, however, the calculated transition zone (TZ) volume was used in the analyses, based on the measured total prostate gland volume rather than the measured TZ volume. The TZ volume was determined by subtracting 20 ml from the total prostate gland volume, as it was assumed that 20 ml was the prostate gland volume at the patient age of forty. Consequently, the measured volume in addition to 20 ml would represent the BPH growth in the TZ, as it has been reported that BPH selectively affects only the TZ of the prostate gland (17A). Thus, it may be argued that the calculated TZ volume in our previous reports did not necessarily reflect the BPH enlargement. In the present study, we measured the volume of both the total prostate gland and the TZ in a subgroup of patients in an effort to validate the results of our previous studies.

At the same time, we re-evaluated the relationships between components of the metabolic syndrome and the BPH growth rate in an extended series of patients. Special attention was paid to the relationship between the insulin level and the development of BPH in order to test the hypothesis that hyperinsulinaemia is causally related to the development of BPH. If this hypothesis is valid, men with metabolic disease and men with components of the metabolic syndrome such as NIDDM, treated hypertension, obesity and dyslipidaemia should have a faster annual BPH growth rate than men without metabolic disease. Moreover, the annual BPH growth rate should increase with increasing fasting plasma insulin levels.

Patients and Methods

Three hundred and seven consecutive patients referred to the Urological Section, Department of Surgery, Varberg Hospital, Varberg, Sweden, with lower urinary tract symptoms were included in this study. Of these patients, 250 had been included in our previous study (16A). One hundred and fourteen of the men, constituting a subgroup, were consecutively subjected to determination of the TZ volume in addition to the total prostate gland volume. Varberg Hospital has a strictly defined catchment area with a permanent population of 135,000 people. The Swedish healthcare system is organized in such a way that the great majority of men living in this area are referred to Varberg Hospital for examination involving potential urological disorders. Most of these men were investigated merely because of lower urinary tract symptoms and needed no medical treatment. A few of them, however, were later on subjected to medical or surgical treatment because of BPH. Men with a known malignant disease, including prostatic cancer, with a significant body weight change (±10 kg) during the last 10 years, on finasterid or alpha-adrenergic blocking medication and men subjected to a previous transurethral resection of the prostate gland with unknown resection weight were excluded.

A patient was said to have had hypertension if this condition was pharmacologically treated and NIDDM if this diagnosis was provided by the patient's medical history. Obesity was defined as a waist measurement of 96 cm or more. Hyperinsulinaemia was said to exist if the fasting plasma insulin value was 9 mU/l or higher. Dyslipidaemia was defined if there was an HDL-cholesterol value of 1.18 mmol/l or lower. A patient was said to have a metabolic disease if he had one or more of the conditions mentioned above.

Data on blood pressure, waist and hip measurements, body height and body weight were collected and body mass index (BMI, kg per m²) and waist/hip ratio (WHR) were calculated. The prostate gland was examined by means of digital rectal examination and ultrasound equipment (B&K Medical 3535). Several ultrasound-guided core biopsies of the prostate gland were taken for histopathological examination if prostatic cancer was suspected, based on digital rectal examination, the transrectal ultrasound findings or a high prostate-specific antigen (PSA) value. The total prostate gland volume and the TZ volume were measured by one and the same experienced examiner by means of ultrasound using the ellipsoid method (18-19A). The non-TZ volume was calculated by subtracting the TZ volume from the total prostate gland volume. The annual BPH growth rate was then calculated. In this calculation, the annual BPH growth rate was based on the assumption that the prostate growth rate is linear over time and that the prostate gland volume was 20 ml when the patient was 40 years old (5A). The following formula was used: total prostate gland volume—20 ml/age—40 years. In the subgroup of 114 patients subjected to both measurement of the total prostate gland volume and the TZ volume, the annual BPH growth rate was calculated using the same formula. The annual TZ growth rate was calculated using the following formula: TZ volume/age—40 years. Thus, it was assumed that the TZ volume was zero at the age of forty, i.e. no BPH growth. The annual growth rate of the non-TZ was calculated using the following formula: total prostate gland volume−TZ volume—20 ml/age—40.

Blood samples were drawn from overnight-fasting patients. Serum for determination of insulin was separated within one hour of sampling and stored at −20° C. until assayed. Fresh serum was analysed for HDL-cholesterol and PSA.

Serum insulin was measured by means of a radioimmunoassay kit, Insulin RIA 100, from Pharmacia Diagnostics, Uppsala, Sweden, using a human insulin standard. HDL-cholesterol was analysed on a Synchrone CX7 instrument from Beckman Instruments Inc, Brea, Calif., USA, with reagents from the same supplier. HDL-cholesterol was measured in the supernatant after precipitation with dextran sulphate and magnesium chloride. The PSA level was measured using Elecsys PSA Immunoassay and Elecsys 1010/2010 Systems (Roche/Boehringer-Mannheim, Germany).

Since most variables in this report were not normally distributed, we preferred to use non-parametric statistics, i.e. the median value, the Spearman correlation coefficient and the Mann-Whitney U-test for the calculations. When performing linear multiple regression analyses, the variables were transformed into logarithms.

The ethical aspects of the study were approved by the Ethical Committee of the Medical Faculty of the University of Göteborg, Sweden

Results

In a subgroup of this study, 114 patients were subjected to determination of the total prostate gland volume, the TZ volume and calculation of the non-TZ volume. The median age of this subgroup was 68 years, range 50-91 years. The median total prostate gland volume was 55 ml, the median TZ volume 35 ml and the median non-TZ of the prostate gland volume was calculated to be 23 ml. The median annual total prostate gland growth rate was 1.27 ml/year, assuming that the total prostate gland volume was 20 ml at the patient age of forty. The median annual TZ growth rate was 1.21 ml/year, assuming that the TZ started to grow from zero at the patient age of forty. The corresponding figure for the calculated non-TZ growth rate was 0.09 ml/year, assuming that the total prostate gland volume was 20 ml at the same age.

Statistically significant correlations were found between the total prostate gland volume and the TZ volume (rs=0.97; p<0.0001) (FIG. 1A) and the calculated non-TZ (rs=0.77; p<0.0001). The TZ volume correlated statistically significantly with the calculated non-TZ volume of the prostate gland (rs=0.59; p<0.0001).

In the total material of 307 patients, the median annual total prostate gland growth rate was 1.03 ml/year, assuming that the prostate gland started to grow from 20 ml at the patient age of forty. The median age in the total material was 71 years, range 48-92 years. The calculated median annual growth rate of the total prostate gland in older men (>71 years) was 1.05 ml/year and in younger men (<71 years) was 1.00 ml/year (n.s). Moreover, age was not statistically significantly correlated with the annual total prostate gland growth rate (rs=−0.05; n.s).

Table 1A shows the median annual BPH growth rates in men without or with metabolic disease and in men with different manifestations of the metabolic syndrome. The median BPH growth rate was faster in men with metabolic disease than in men without metabolic disease. Moreover, men with NIDDM, treated hypertension, obesity and/or dyslipidaemia had a highly statistically faster median annual BPH growth rate than men without metabolic disease. Statistically significant correlations were found between the annual BPH growth rate and the following variables of the metabolic syndrome: diastolic blood pressure (rs=0.27; p<0.001), body weight (rs=0.27; p<0.001), BMI (rs=0.22; p<0.001), waist measurement (rs=0.30; p<0.001), hip measurement (rs=0.22; p<0.001), WHR (rs=0.20; p<0.001), and HDL-cholesterol (rs=−0.15; p<0.001).

In Table 2A, the fasting plasma insulin levels have been dichotomized. Patients with a fasting plasma insulin level lower than 9 mU/l were compared with patients with a higher fasting plasma insulin level. Men with a higher fasting plasma insulin level had a faster BPH growth rate than men with a lower fasting plasma insulin level.

In Table 3A, data are given for the quartiles of the patient material. The first quartile represents data on patients with a fasting plasma insulin level lower than 7 mU/l. The second quartile consists of patients with a fasting plasma insulin level between 7 and 9 mU/l. The third quartile shows data on patients with a fasting plasma insulin level between 9 and 13 mU/l and the fourth quartile gives data on patients with a fasting plasma insulin level higher than 13 mU/ml. In the comparison between the first and the fourth quartiles, the BPH growth rate was faster in the fourth quartile.

A statistically significant correlation was found between the fasting plasma insulin level and the annual BPH growth rate (rs=0.16; p=0.009) and the total prostate gland volume (rs=0.18; p=0.003). When performing a multiple regression analysis, we used the total prostate gland volume instead of the annual BPH growth rate as the dependent variable in order to include age in the analysis. In this analysis, fasting plasma insulin (p<0.001) and age p<0.001) had significant effects on the total prostate gland volume. Including more independent variables would not have been meaningful considering the statistically significant correlations between the variables mentioned above.

Discussion

Transrectal ultrasound using the elipsoid method is an accepted way of measuring the total prostate gland volume and the BPH volume (18A-19A). It has been claimed that BPH occurs exclusively in the TZ (17A). If this is true, the results of the present report clearly show that the total prostate gland volume, which in our studies has been used as a measure of BPH, is an adequate exponent of BPH, as the correlation coefficient between the total prostate gland volume and the TZ volume was rs=0.97; p<0.0001 (FIG. 1A). This means that the validity coefficient is 0.97, i.e. an almost perfect correspondance between the total prostate gland volume and the TZ volume.

It could not be confirmed in the present report, however, that BPH occurs exclusively in the TZ, as the calculated volume of the non-TZ increased in size in relation to the TZ volume and the total prostate gland volume. It may be argued that the median annual non-TZ growth rate was rather low, about 0.09 ml/year. In some men with the largest non-TZ volume (>30 ml), however, the median annual non-TZ growth rate was 0.67 ml/year. If these findings are true, this means that with increasing age, not only does the TZ grow, but also the non-TZ of the prostate gland, although at a slower rate. These findings seem to extend BPH to involve the whole prostate gland.

The highly significant intercorrelations between the total prostate gland volume, the TZ volume and the non-TZ volume suggest that the causal factor in BPH is a systemic rather than a local factor. Our data seem to suggest that the TZ is more sensitive to this causal systemic factor than the non-TZ.

The main conclusion of our findings regarding volume measurements of the prostate gland can be summarized as follows: The total prostate gland volume, rather than the TZ volume, seems to be the most appropriate BPH expression when exploring the metabolic profile of men with fast-growing BPH compared with men with slow-growing BPH. Thus, this conclusion validates the results of the present and our previous reports (15A-16A).

In previous studies, we have hypothesized that hyperinsulinaemia is the systemic causal factor in the development of BPH (15A-16A). Hyperinsulinaemia is one of the components of the metabolic syndrome. This syndrome has been described as a single entity characterized by a defect in the insulin-mediated glucose uptake (20A-21A). The primary metabolic abnormality is mainly localized to the muscle, the adipose tissue and the liver of these patients, leading to an insulin-resistance and a secondary hyperinsulinaemia (23A). The hypothesis that hyperinsulinaemia is causally related to the development of BPH is supported by the following findings in the present study.

Firstly, it is obvious from Table I that there is a relationship between the occurrence of metabolic aberrations which are known to be associated with hyperinsulinemia (20A-22A) on the one hand and the annual BPH growth rate on the other. The reason for this is that men with metabolic disease have a faster median annual BPH growth rate than men without any metabolic disease. Moreover, men with diagnosed manifestations of the metabolic syndrome, such as NIDDM, treated hypertension, obesity and dyslipidaemia all had a faster annual BPH growth rate than men without any manifestation of the metabolic disease. Moreover, there were highly significant positive correlations between the diastolic blood pressure, several expressions of obesity such as BMI, waist measurement, hip measurement and WHR on the one hand and the annual BPH growth rate on the other. There was also a highly negative correlation between the HDL-cholesterol level and the annual BPH growth rate. All these conditions of the metabolic syndrome have clearly been established to be associated with hyperinsulinaemia (20A-22A). Consequently, these findings support the hypothesis of a causal relationship between hyperinsulinaemia and the development of BPH. The median annual BPH growth rate in men without metabolic disease in the present report was 0.72 ml/year. The corresponding median annual BPH growth rate in healthy men of the same age, given in another report, was 0.40 ml/year (5A). Assuming that there is a relationship between the metabolic aberrations and the annual BPH growth rate, this comparison implies that the patients included in the present report as men without metabolic disease actually had a metabolic disorder.

Secondly, men with a high fasting plasma insulin level had a faster median annual BPH growth rate than men with a low fasting plasma insulin level. When the patients were divided into quartiles, the median annual BPH growth rate in men with the lowest insulin level was 0.84 ml/year. The corresponding median annual growth rate in men with the highest insulin level was 1.49 ml/year. In the two middle quartiles, i.e. the second and the third, the median annual BPH growth rates were 1.00 ml/year and 1.07, respectively (n.s.).

Thirdly, there is a highly significant correlation between the fasting plasma insulin level and the annual BPH growth rate. It may be argued that the correlation coefficient between the fasting plasma insulin level and the annual BPH growth rate was only 0.16 (p=0.009). However, it must be realised that the prostate gland volume at the age of approximately 70 years is the result of a process which has been going on for about 30 years. The plasma insulin value available in the present report, on the other hand, reflects the insulin level one of the mornings at the end of this period.

Finally, when performing a multivariate analysis using fasting plasma insulin and age as covariants related to the total prostate gland volume, the correlation between the insulin level and the total prostate gland volume became statistically significant.

Assuming a causal role of hyperinsulinaemia in the development of BPH, this means that minor reductions in insulin levels might bring about significantly lower BPH growth rates and prostate gland volumes. These minor reductions in insulin levels might be generated by measures such as weight reduction (24A-25A), dietary changes (26A-27A) or increased physical activity (28A-31A) or a combination of measures such as increased physical activity and changed dietary regulation (32A-33A) or modification of the diet and smoking habits (34A). In fact, recent epidemiological studies have confirmed that self-reported lifestyle factors such as increased physical activity (35A), avoidance of smoking (36A) and intake of fruit (37A) appear to protect against development of BPH. Some drugs available today are also known to decrease the insulin levels and improve insulin sensitivity, such as metformin (38A), captopril (39A), prazosin (40A-41A), dilevalol (42A) and doxazosin (43A-45A).

The data in the present report also generate a hypothesis of an increased sympathetic nerve activity in men with fast-growing BPH. The reason for this is that hypertension (46A-48A), obesity (49A-50A) and hyperinsulinaemia (51A-53A) are conditions, which all have been claimed to have an increased sympathetic nerve activity. There is mounting evidence that the line between these conditions and the increased sympathetic nerve activity is the sympatho-excitatory effects of insulin (54A). It has been shown that insulin has a stimulating effect on the ventromedial hypothalamic nucleus that regulates the sympathetic nervous system (55A). Induced hyperinsulinaemia has been shown to increase the catecholamine levels in plasma and tissues (54A). Hyperinsulinaemia has also been shown to increase the impulse flow in peripheral sympathetic nerves (56A). Thus, it seems reasonable to believe that men with fast-growing BPH and hypertension, obesity and an elevated insulin level as risk factors in accordance with our findings, might have an increased sympathetic nerve activity.

It has recently been reported that alpha-receptor blockers as a class are able to change the prostate growth equilibrium by inducing apoptosis of both epithelial and stromal smooth muscle cells (57A). In this study, it has been hypothesised that catecholamines might have a trophic effect on the growth of prostatic cells by slowing down the apoptotic process, offering a possible mechanism by which the hyperinsulinaemia and the sympathetic nerve system are involved in the aetiology of BPH.

In the clinical setting, our findings mean that any doctor facing a patient with BPH should consider the possible presence of NIDDM, hypertension, obesity, high insulin or low HDL-cholesterol levels in that patient. Conversely, in patients with these conditions, the possibility of a clinically important BPH should be kept in mind, as the prostate gland volume has been shown to correlate positively with the severity of lower urinary tract symptoms and negatively with the peak urinary flow rate (58A).

In conclusion, the results of the present report suggest that the total prostate gland volume is a valid expression of BPH when exploring the metabolic profile in men with BPH. Moreover, the hypothesis generated in two previous studies that hyperinsulinaemia is causally related to the development of BPH is supported in the present series of patients (15A-16A). It might be speculated that this hyperinsulinaemia induces lower urinary tract symptoms by two entirely different mechanisms. Firstly, the trophic effect of hyperinsulinaemia induces an enlarged prostate gland and an obstructed urinary flow. Secondly, through its sympatho-excitatory effect, this hyperinsulinaemia adds a dynamic component to the obstructed urinary flow. If these speculations are true, it might be possible to develop effective preventive and therapeutic strategies which might prevent the progression of the BPH and reduce the need for surgery with methods available today.

TABLE 1A
The median annual BPH growth rate in men without or with
metabolic disease and in men with different manifestations
of the metabolic syndrome.

| | | | Median | P-value versus |
|---|---|---|---|---|
| | | | annual BPH | men without |
| | | | growth rate, | metabolic |
| | n | Age | ml/year | disease |
| Without metabolic disease | 60 | 68 | 0.72 | |
| With metabolic disease | 247 | 72 | 1.13 | <0.0001 |
| NIDDM | 39 | 74 | 1.49 | <0.0001 |
| Treated hypertension | 68 | 74 | 1.13 | <0.0001 |
| Obesity | 145 | 72 | 1.36 | <0.0001 |
| Dyslipidaemia | 139 | 72 | 1.11 | <0.0001 |

Metabolic disease = men with one or more of the conditions: NIDDM, treated hypertension, obesity, high insulin and low HDL-cholesterol level;
NIDDM = non-insulin-dependent diabetes mellitus;

TABLE 2A
The median age, median total prostate gland volume and the
median annual BPH growth rate in men with high or low fasting
plasma insulin values (dichotomies).

| | Fasting plasma insulin, mU/l | | |
|---|---|---|---|
| | <9 n = 142 | ≧9 n = 138 | P-value |
| Age, years | 70 | 72 | 0.61 |
| Prostate volume, ml | 46 | 53 | 0.012 |
| Annual BPH growth rate, ml/year | 0.93 | 1.16 | 0.019 |

TABLE 3A
The median age, median total prostate gland volume and the
median annual BPH growth rate in men with different fasting plasma
insulin levels (quartiles).

| | I | II | III | IV | |
|---|---|---|---|---|---|
| Fasting plasma insulin | <7 | 7-9 | 9-13 | >13 | P-value |
| level, mU/l | n = 82 | n = 60 | n = 69 | n = 69 | I vs. IV |
| Age, years | 69 | 72 | 72 | 72 | 0.39 |
| Prostate volume, ml | 45 | 50 | 48 | 61 | 0.009 |
| Annual BPH growth rate, | 0.84 | 1.00 | 1.07 | 1.49 | 0.015 |
| ml/year | | | | | |

### REFERENCES

1A. Barry, M J.: Epidemiology and natural history of benign prostatic hyperplasia. Ur Clin. North Am., 1990; 17:495-507

2A. Partin A W, Oesterling J E, Epstein J I, Horton R and Walsh P. Influence of age ar endocrine factors on the volume of benign prostatic hyperplasia J. Urol. 1991; 145:40 9.

3A. Guess H A. Benign prostatic hyperplasia: Antecedents and Natural Histor Epidemiological Reviews 1992; 14:131-53.

4A. Oesterling J. Benign prostatic hyperplasia: Medical and minimally invasive treatme options. New Engl. J. Med 1995; 332:99-109

5A. Berry S J, Coffey D S, Walsh P C and Ewing L L. The development of human benig prostatic hyperplasia with age. J. Urol. 1984; 132:474-9.

6. Walsh P C. Benign prostatic hyperplasia. In: Campbell's Urology. Edited by Walsh C., Retik A. B., Stamey T. A. and Vaughan Jr. E. D. Philadelphia: W. B. Saunders, 1009-1027, 1992

7A. Horton R, Hsieh P, Barberia J, Pages L and Cosgrove M. Altered blood androgens elderly men with prostate hyperplasia J. Clin. Endocrinol. Metab. 1975; 41:793-6.

8A. Vermeulen A and De Sy W. Androgens in patients with benign prostatic hyperplas before and after prostatectomy. J. Clin. Endocrinol. Metab. 1976; 43:1250-4.

9A. Ishimaru T, Pages L and Horton R. Altered metabolism of androgens in elderly me with benign prostatic hyperplasia J. Clin. Endocrinol. Metab. 1977; 45:695-701.

10A. Hammond G L, Kontturi M, Vihko P and Vihko R. Serum steroids in normal males a patients with prostatic diseases. Clin. Endocrinol. 1978; 9:113-21.

11A. Bartsch W, Becker H, Pinkenburg F A and Krieg M. Hormone blood levels and the inter-relationships in normal men and men with benign prostatic hyperplasia (BPH Acta Endocrinol. 1979; 90:727-36.

12A. Lukkarinen O. Total and SHBG-bound testosterone and 5 alpha-dihydrotestostero serum concentrations in normal elderly men and patients with benign prostat hypertrophy before and after removal of the adenoma. Brit. J. Urol. 1980; 52:377-80.

13A. Drafta D, Proca E, Zamfir V, Schindler A E, Neacsu E and Stroe E. Plasma steroids benign prostatic hypertrophy and carcinoma of the prostate. J. Steroid. Biochem. 198 17:689-93.

14A. Brochu M and Belanger A. Comparative study of plasma steroid and steroid glucoroni levels in normal men and in men with benign prostatic hyperplasia. The Prostate 198 11:33-40.

15A. Hammarsten J, Högstedt B, Holthuis N and Mellström D. Components of the metabol syndrome—risk factors for the development of benign prostatic hyperplasia Prosta cancer and prostatic diseases 1998; 1:157-62.

16A. Hammarsten J and Högstedt B. Clinical, anthropometric, metabolic and insulin profi of men with fast annual growth rates of benign prostatic hyperplasia Blood pressu 1999; 8:29-36

17A. McNeal J. Pathology of benign prostatic hyperplasia Urol. Clin. N. Am. 1990; 17:477-86

18A. Littrup P J, Williams C R, Egglin T K and Kane R A. Determination of prostate volume with transrectal US for cancer screening—Part II. Accuracy of in vitro and in vivo techniques. Radiology 1991; 179:49-53.

19A. Terris M K and Stamey T A. Determination of prostate volume by transrectal ultrasound J. Urol. 1991; 145:984-7.

20A. DeFronzo R A and Ferrannini E. Insulin resistance. A multifaceted syndrome responsible for NIDDM, obesity, hypertension, dyslipidemia, and atherosclerotic cardiovascular disease. Diabetes Care 1991;14:173-94

21A. Reavan G M Banting lecture 1988. Role of insulin resistance in human disease. Diabet 1988; 37:1595-1607.

22A. Rett K, Wicklmayr M and Mehnert H. New aspects of insulin resistance in hypertensio European Heart Journal 1994; 15 (Supplement C):78-81

23A. Krotkiewski M. Role of muscle capillarization and morphology in the development insulin resistance and metabolic syndrome. Presse Med. 1994; 23:1353-6

24A. Kalkhoff R K, Kim H S, Cerletty J and Ferrou C A. Metabolic effects of weight loss obese subjects. Changes in plasma substrate levels, insulin and growth hormo responses. Diabetes 1971; 20:83-91

25A. Jimenez J, Zuniga-Guajardo S, Zinman B and Angel A. Effects of weight loss massive obesity on insulin and c-peptide dynamics: sequential changes in insuli production, clearance, and sensitivity. Endocrinol Metab. 1987; 64:661-8

26A. Haber G B, Heaton K W, Murphy D and Burroughs L F. Depletion and disruption dietary fibre: effects on satiety, plasma-glucose, and serum-insulin. Lancet 1977; ii:67 82

27A. Karlström B, Vessby B, Asp N-G and Ytterfors A. Effects of four meals with differed kinds of dietary fibre on glucose metabolism in healthy subjects and non-insuli dependent diabetic patients. European Journal of Clinical Nutrition 1988; 42:519-526

28A. Björntorp P, De Jounge K, Sjöström L and Sullivan L. The effect of physical training insulin production in obesity. Metabolism 1970; 19:631-38.

29A. Krotkiewski M, Bylund-Fallenius A-C, Holm J, Björntorp P, Grimby G ar Mandroukas K. Relationship between muscle morphology and metabolism in obe women: the effects of long-term physical training. Europ. J. Clin. Invest. 1983; 13:5-12

30A. Seals D R, Hagberg J M, Hurley B F, Ehsani A A and Holloszy J. O. Effects of enduran training on glucose tolerance and plasma lipid levels in older men and women. JAM 1984; 252:645-9.

31A. DeFronzo R A, Sherwin R S and Kraemer N. Effect of physical training on insulin actic in obesity. Diabetes 1987; 36:1379-85

32A. Nilsson P M, Lindholm L H and Schersten B F. Life style changes improve insul resistance in hyperinsulinaemic subjects: a one-year intervention study of hypertensive and normotensives in Dalby. J. Hypertens. 1992; 10:1071-78.

33A. Eriksson K-F and Lindgärde F. Prevention of type 2 (non-insulin-dependent) diabet mellitus by diet and physical exercise. The 6-year Malmö feasibility study. Diabetolog 1991; 34:891-8.

34A. Hjermann I, Leren P, Norman N, Helgeland A and Holme I. Serum insulin response oral glucose load during a dietary intervention trail in healthy coronary high risk me the Oslo study. Scand. J. Clin. Lab. Invest. 1980; 40:89-94

35A. Platz E A, Kawachi I, Rimm E B, Colditz G A, Stampfer M J, Willett W C ar Giovannucci E. Physical activity and benign prostatic hyperplasia. Arch. Int. Med. 199 158:2349-56

36A. Platz E A, Rimm E B, Kawachi I, Colditz G A, Stampfer M J, Willett W C ar Giovannucci E. Alcohol consumption, cigarette smoking, and risk of benign prostat hyperplasia. Am. J. Epidemiol 1999; 149:106-15.

37A. Lagiou P, Wuu J, Trichopoulou A, Hsieh C-C, Adami H-O and Trichopoulos D. Di and benign prostatic hyperplasia: A study in Greece. Urology 1999; 54:284-290.

38A Landin K, Tengborn L and Smith U. Treating insulin resistance in hypertension wi metformin reduces both blood pressure and metabolic risk factors. J. Intern. Med. 199 229:181-87

39A. Pollare T, Lithell H and Berne C. A comparison of the effects of hydrochlorothiazi and captopril on glucose and lipid metabolism in patients with hypertension. N. Engl. Med. 1989; 321:868-73.

40A. Pollare T, Lithell H, Selinus I and Berne C. Application of prazosin is associated with increase of insulin sensitivity in obese patients with hypertension. Diabetologia 198 31:415-20

41A. Swislocki A L M, Hoffman B B, Sheu W H-H, Chen Y-D I. and Reaven G M. Effect prozasin treatment on carbohydrate and lipoprotein metabolism in patients wi hypertension. Am. J. Med. 1989; 86 (Suppl.1B):14-8.

42A. Haenni A and Lithell H Treatment with a beta-blocker with beta2-agonism improve glucose and lipid metabolism in essential hypertension. Metabolism 1994: 43:455-61.

43A. Andersson P-E, Johansson J, Berne C and Lithell H. Effects of selective alpha1 ar beta1 adrenoreceptor blockade on lipoprotein and carbohydrate metabolism i hypertensive subjects, with special emphasis on insulin sensitivity. J. Huma Hypertens. 1994; 8:219-26.

44A Andersson P-E and Lithell H. Metabolic effects of doxazosin and enalapril i hypertriglyceridemic, hypertensive men. Relationship to changes in skeletal musc blood flow. Am. J. Hypertens. 1996; 9:323-33

45A. Shieh S-M, Sheu W H-H, Shen D-C, Fuh M M-T, Chen Y-D I. and Reaven G Glucose, insulin, and lipid metabolism in doxazosin-treated patients with hypertension Am. J. Hypertens. 1992; 5:827-31.

46A. Yamada Y, Miyajima E, Tochikubo O, Matsukawa T and Ishii M. Age-related change in muscle sympathetic nerve activity in essential hypertension. Hypertension. 198 13:870-7

47A. Anderson E A, Sinkey C A, Lawton W J. and Mark A L. Elevated sympathetic ner activity in borderline hypertensive humans. Evidence from direct intraneural recording Hypertension 1989; 14:177-83.

48A. Floras J S and Hara K. Sympathoneural and haemodynamic characteristics of your subjects with mild essential hypertension. J. Hypertens. 1993; 11:647-55

49A. Troisi R J, Weiss S T, Parker D R, Sparrow D, Young J B and Landsberg L. Relation obesity and diet to sympathetic nervous system activity. Hypertension 1991; 17:669-77

50A. Grassi G, Seravalle G, Cattaneo B M, Bolla G B, Lanfranchi A, Colombo Giannattasio C and Brunani A. Sympathetic activation in obese normotensive subject Hypertension 1995; 25(part 1):560-3

51A. Anderson E A, Hoffman R P, Balon T W, Sinkey C A and Mark AL. Hyperinsulinem produces both sympathetic neural activation and vasodilatation in normal humans. Clin. Invest. 1991; 87:2246-52.

52A. Berne C, Fagius J, Pollare T and Hjemdahl P. The sympathetic response to euglycaem hyperinsulinemia: evidence from microelectrode nerve recordings in healthy subject Diabetologia 1992; 35:873-79

53A. Rowe J W, Young J B, Minaker K L, Stevens A L, Pallotta J and Landsberg L. Effects insulin and glucose infusions on sympathetic nervous system activity in normal ma Diabetes 1981; 30:219-25

54A. Morgan D A, Balon T W, Ginsberg B H and Mark A L. Nonuniform regional sympathet nerve responses to hyperinsulinemia in rats. Am. J. Physiol. 1993; 264(Regulato Integrative comp. Physiol. 33): R423-R427

55A. Landsberg L. Diet, obesity and hypertension: An hypothesis involving insulin, t sympathetic nervous system, and adaptive thermogenesis. Q. J. Med. 1986; 236:1081-9

56A. Berne C, Pollare T and Fagius J. The sympathetic outflow in vasoconstrictor ner fascicles to muscle is increased during euglycaemic hyperinsulinaemia. Diabetolog 1989; 32:465A

57A. Chon J K, Borkowski A, Partin A W, Isaacs J T, Jacobs S C and Kyprianou N. Alpha adrenoceptor antagonists terazosin and doxazosin induce prostate apoptosis witho affecting cell proliferation in patients with benign prostatic hyperplasia. J. Urol. 199 161: 2002-2008.

58A. Girman C J, Jacobsen S J, Guess H A, Oesterling J E, Chute C G, Panser L A and Lieb M M. Natural history of prostatism: Relationship among symptoms, prostate volume ar peak urinary flow rate. J. Urol. 1995; 153:1510-5.

Study 2: Calculated Fast-Growing Benign Prostatic Hyperplasia—a Risk Factor for Developing Clinical Prostate Cancer

Benign prostatic hyperplasia (BPH) and clinical prostate cancer are very common in elderly men in the Western society (1B, 2B). Both conditions are neoplastic and are characterized by an increased growth rate of the prostatic epithelium. BPH predominantly develops within the transition zone, while clinical prostate cancer most commonly arises within the peripheral zone of the prostate gland (3B, 4B). Whether there is an association between the development of BPH and clinical prostate cancer is still controversial.

Two studies based on prostate gland volume assessment by digital rectal examination have suggested an increased risk of clinical prostate cancer in patients with BPH. Armenian et al. have reported that patients with BPH run a risk of developing prostate cancer—a risk several times greater than that of men without BPH (5B). The other study was a representative and prospective study from Gothenburg comprising 1,023 men, all of whom were born in 1913 (6B). In 1967, these men were examined by digital rectal examination with regard to BPH by an experienced internist. On this occasion, 278 of them were considered to have BPH. Twenty years later, 6.7% of these men had been diagnosed as having prostate cancer, compared with 3.9% in the group without BPH (P<0.03). These studies can be criticized, however, since the diagnosis of BPH was made on clinical rather than on histological grounds using volume values lacking in accuracy.

Conflicting conclusions have been reported in two other studies including men undergoing transurethral resection of the prostate (TURP) and a control group not undergoing this surgical procedure. The relative risk for the development of prostate cancer in the BPH group was found to be 0.88 (n.s.) and 0.80 (n.s.) after 10 years in the two studies, respectively (7B, 8B). These studies could be criticized, however, since BPH might predispose to prostate cancer in proportion to the amount of excess BPH tissue and TURP might reduce the risk of developing subsequent clinical prostate cancer, just as breast reduction procedures reduce the risk of breast cancer development in women (9B).

In the present report, the hypothesis that there is an association between development of BPH and clinical prostate cancer has been tested on 220 men with recently diagnosed clinical prostate cancer. If this hypothesis is true, the group of men with fast-growing BPH would have more pronounced clinical prostate cancer than the group of men with slow-growing BPH.

Patients and Methods

Two hundred and twenty patients referred to the Urological Section, Department of Surgery, Varberg Hospital, Varberg, Sweden, in whom clinical prostate cancer was diagnosed, were consecutively included in this study. Clinical prostate cancer was defined as a prostate tumor indicated by a digital rectal examination or by ultrasound and verified histopathologically using the technique of transrectal ultrasound-guided automatic needle biopsy of the prostate gland. The core biopsy was morphologically classified as well differerentiated (G1), moderately differentiated (G2) or poorly differentiated (G3) cancer by our histopathologists. The grading of the histopathology has been performed according to Gleason (10B). The prostate cancer tumors were also subjected to clinical staging and classified in accordance with the 1992 TNM classification (11B). Men with clinical prostate cancer T2-3 and PSA<50 ng/mL were compared with a group of men with clinical prostate cancer T3 and PSA≧50 ng/mL. The reason for separating men with clinical prostate cancer into one group with PSA<50 ng/mL from another group with clinical prostate cancer, PSA≧50 ng/mL, was that we wanted to study the metabolic factors that are promoters of the development of clinical prostate cancer. It is well recognized that men suffering from advanced prostate cancer lose appetite and weight and, later on, develop a reduction in the fasting plasma insulin level and the blood pressure. Thus, these factors should not be considered in men with advanced clinical prostate cancer. On the other hand, men with clinical prostate cancer, PSA≧50 ng/mL, could be included when studying more stable factors, such as the prevalence of atherosclerosis, NIDDM and treated hypertension, body tallness and prostate cancer-related factors, such as prostate cancer stage, grade and PSA-level. Most of the 220 men were investigated because of symptoms generated by the prostate cancer tumors, but some had their prostate cancer discovered while seeking medical advice for other reasons. The median age of the 220 men was 74 years (range 49-91 years).

Varberg Hospital has a strictly defined catchment area with a permanent population of 140,000 people. The Swedish health care system is organized in such a way that a great majority of men living in this area are referred to Varberg Hospital for medical care, including urologic problems.

Men with another malignant disease in their medical history, men with a significant body weight change (±10 kg) during the last 10 years, men on finasterid medication and men subjected to a previous transurethral resection of the prostate with unknown resection weight were excluded. Moreover, men subjected to hormonal manipulation (ablation of the testes, treatment with GmRH analogues, antiandrogens, testosterone, estrogens, steroids, insulin, thyroxin or growth hormone) were excluded. In this study, only the clinical cancer tumors, T2-T3, Nx, Mx were included because it was considered that inclusion of more pronounced tumors could distort the evaluation of anthropometric, hormonal and metabolic data. It is obvious that men suffering from pronounced prostate cancer lose appetite and weight, which would affect several risk factors we wanted to study in the present study. The largest prostate cancer tumor included in the present study, as estimated by digital rectal examination and ultrasound, had a diameter of 2 cm, which gives a maximal volume of 4.2 ml.

A patient was said to have had hypertension if this condition had been pharmacologically treated and non-insulin dependent diabetes mellitus (NIDDM) if this diagnosis was provided by the patient's medical records. Atherosclerotic disease manifestations include coronary artery disease, cerebrovascular disease and peripheral arterial insufficiency. Coronary artery disease includes symptoms of effort angina pectoris and a history of myocardial infarction. Cerebrovascular disease is defined as a history of stroke or a transient ischemic attack (TIA) documented by the patient file. Patients with a history of arterial aneurysm, intermittent claudication, rest pain or peripheral gangrene caused by arterial insufficiency—whether subjected to surgery or not—were defined as patients with peripheral arterial insufficiency.

Data on blood pressure, waist and hip measure, body tallness and body weight were collected and the body mass index (BMI, kg per m²) and the waist/hip ratio (WHR) were calculated. The prostate gland was examined using digital rectal examination and ultrasound equipment (B&K Medical 3535). The prostate gland volume was determined by means of ultrasound using the ellipsoid method (12B, 13B). The age-adjusted prostate growth rate was calculated. In this calculation, the annual BPH growth rate was based on the assumption that the prostate growth rate is linear over time and that the prostate gland volume is 20 mL when the patient is 40 years old (14B). The following formula was used: total prostate gland volume—20 mL/age—40 years.

Blood samples were drawn from overnight-fasting patients. Serum to determine insulin was separated within one hour of sampling and stored at −20° C. until assayed. Fresh serum was analysed for total cholesterol, HDL-cholesterol, triglycerides, uric acid and alanine aminotransferas (ALAT, EC 2.6.1.2.).

Serum insulin was measured by means of a radioimmunoassay kit, Insulin RIA 100, from Pharmacia Diagnostics, Uppsala, Sweden, using a human insulin standard. Total cholesterol, HDL-cholesterol, triglycerides, uric acid and ALAT were analyzed on a Synchrone CX7 instrument from Beckman Instruments Inc, Brea, Calif., USA, with reagents from the same supplier. HDL-cholesterol was measured in the supernatant after precipitation with dextran sulphate and magnesium chloride. LDL-cholesterol was calculated using the Friedewald formula. Total PSA was measured by means of Elecsys total PSA Immunoassay from Roche.

Since most variables in this report were not normally distributed, we preferred to use non-parametric statistics, i.e. the median value and the Mann-Whitney U-test for calculations of differences between groups and the Chi-square or the Fisher test for calculations of differences in proportions between groups. The dichotornisation of the variables has been performed using the median values. In the present report, a risk factor was defined as a factor that is statistically associated with the occurrence of a disease (15B).

The ethical aspects of the study were approved by the Ethical Committee of the Medical Faculty of the University of Göteborg, Sweden.

Results

The median annual BPH growth rate was 1.07 mL/year in men with clinical prostate cancer, PSA<50 ng/mL, and 1.15 mL/year in the total material including men with clinical prostate cancer, PSA≧50 ng/mL, assuming that the BPH started to develop in the patients at the age of forty.

In Table 1B, the annual BPH growth rates have been dichotomized in men with clinical prostate cancer, PSA<50 ng/mL, excluding men with clinical prostate cancer, PSA≧50 ng/mL. Patients with an annual BPH growth rate lower than 1.07 mL/year were compared with patients with a higher BPH growth rate. The patients with fast-growing BPH had a higher systolic and diastolic blood pressure than men with slow-growing BPH. The patients in the former group were also taller and more obese, as determined by body weight, BMI, waist and hip measurements. Men with fast-growing BPH had a higher fasting plasma insulin level and a borderline significance of lower HDL-cholesterol levels. Moreover, men with fast-growing BPH had a more pronounced prostate cancer as measured by grade and PSA level.

Table 2B gives the data on the total material of men with clinical prostate cancer, including men with PSA≧50 ng/mL. Patients with an annual BPH growth rate lower than 1.15 mL/year were compared with patients with a higher BPH growth rate. In this comparison, the differences between slow and fast-growing BPH were as follows. The prevalence of NIDDM was higher in men with fast-growing BPH than in men with slow-growing BPH. Moreover, men with fast-growing BPH showed a borderline significance as regards higher stage. The statistical significance levels between the groups of men with slow-growing or fast-growing BPH concerning grade and the PSA level were increased. On the other hand, the statistical significance levels between men with slow-growing or fast-growing BPH were decreased or disappeared when it came to systolic and diastolic blood pressure and obesity, as determined by body weight, BMI, waist and hip measurement. Moreover, the above-mentioned statistically significant difference in fasting plasma insulin disappeared.

Discussion

Previous studies have provided evidence that fast-growing BPH is a risk factor for NIDDM, hypertension, tallness, obesity, dyslipidaemia and hyperinsulinaemia (16B, 17B, 18B). These studies have also suggested that fast-growing BPH is a component of the metabolic syndrome and that patients with fast-growing BPH might share the same metabolic abnormality of a defective insulin-mediated glucose uptake and a secondary hyperinsulinaemia as patients with the metabolic syndrome. This syndrome has been described as a single entity characterized by a defect in the insulin-mediated glucose uptake (19B, 20B, B21). The primary metabolic abnormality of the metabolic syndrome is mainly localized to the muscle, the adipose tissue and the liver of the patients suffering from this syndrome, leading to an insulin-resistance and a secondary hyperinsulinaemia (22B). In the present study, men with recently diagnosed clinical prostate cancer and fast-growing BPH had the same clinical, anthropometric, metabolic and insulin profiles as were found in men with fast-growing BPH without clinical prostate cancer in our previous studies. This confirms the conclusion in our previous reports that BPH is a facet of the metabolic syndrome (16B, 17B, 18B).

The most important finding in the present study, however, was that men with recently diagnosed clinical prostate cancer and with fast-growing BPH had a more pronounced prostate cancer as shown by a higher stage and grade of the clinical prostate cancer and a higher PSA level than men with a clinical prostate cancer and slow-growing BPH. It may be argued that the higher tumor stage only showed a borderline significance and that the higher PSA level could be explained by an increased BPH volume among men with fast-growing BPH. However, the fact that three independent observations go in the same direction strongly supports the conclusion that men with fast-growing BPH have a heavier cancer load, which means a poorer prognosis than men with slow-growing BPH. Our findings suggest that fast-growing BPH is a risk factor for the development of clinical prostate cancer. Thus, our data generate a hypothesis that clinical prostate cancer, besides NIDDM, hypertension, obesity, dyslipidaemia and BPH, is a component of the metabolic syndrome and that clinical prostate cancer patients may share the same metabolic abnormality of a defective insulin-mediated glucose uptake and a secondary hyperinsulinaemia as patients with the metabolic syndrome. Moreover, our data generate a hypothesis that hyperinsulinaemia is a promoter of clinical prostate cancer.

It has been claimed that clinical prostate cancer belongs to a group of diseases referred to as “Western diseases”—because of their high prevalence in affluent Western countries and regions of Europe and North America, compared with Asian countries (26B). This group of diseases also includes atherosclerotic disease manifestations, NIDDM, hypertension, obesity and dyslipidaemia. The results of the present study support the concept that clinical prostate cancer belongs to this group of diseases. The results of the present study also offer a hypothetical mechanism—hyperinsulinaemia—by which clinical prostate cancer could be linked to the “Western diseases”.

If the conclusions in the present report hold true, it means in the clinical setting, that any doctor facing a male patient with fast-growing BPH, NIDDM, hypertension, obesity or/and dyslipidaemia, should consider the possibility that the patient has clinical prostate cancer. Moreover, the presence of one or several of these conditions in a man with clinical prostate cancer could mean a heavier tumor load and a poorer prognosis. Conversely, in men with recently diagnosed prostate cancer, the possible presence of NIDDM, hypertension, obesity and/or dyslipidaemia should be considered. Assuming a promoting role of insulin in developing clinical prostate cancer, it might be speculated that minor reductions in insulin levels might bring about a significantly lower growth rate of clinical prostate cancer. This would make primary, secondary and tertiary prevention possible. These minor reductions in insulin levels might be generated by factors such as weight reduction (23B, 25B), dietary changes (27B, 28B) or increased physical activity (29B-32B) or a combination of factors, such as increased physical activity and changed dietary regulation (24B, 33B) or modification of the diet and smoking habits (34B). Some drugs available today are also known to decrease the insulin levels and improve insulin sensitivity, such as metformin (35B), captopril (36B), prazosin (37B, 38B), dilevalol (39B) and doxazosin (40B-42B).

The present report does not primarily concern the exact extent of the clinical prostate cancer, but the association between the BPH-growth rate and the stage, grade and PSA-level of clinical prostate cancer. The BPH-growth rate may be looked upon as a measure of the metabolic abnormality. However, it must be concluded that the cancer growth per se did not have any impact on the BPH growth rate and that the pronounced cancer disease did not affect the metabolism of the patients.

As regards the impact of the prostate cancer growth on the BPH growth rate, we excluded all patients in whom the local cancer growth within the prostate gland was so extensive that it could have a significant influence on the BPH growth rate. Consequently, we excluded all clinical prostate cancers with a diameter of more than 2 cm. as measured by digital rectal examination and ultrasound. This diameter corresponds to a volume of 4.2 mL, which should be compared with the median total prostate gland volume in the present report of 39 mL and 75 mL in the groups with slow and fast-growing BPH, respectively. This means that the cancer growth had only an insignificant effect on the BPH growth rate.

As for the impact of the prostate cancer on the patient's metabolism, it is clear that men suffering from advanced prostate cancer lose appetite and weight. It is well recognized that a weight reduction in man is accompanied by a decreased insulin level and blood pressure (23B, 24B, 25B). Consequently, these factors were excluded at the comparison between the slow-growing and fast-growing BPH-groups when patients with high PSA-values (≧50 ng/mL) were included. On the other hand, the inclusion of patients with high PSA values (>50 ng/mL) should not distort such factors as the prevalence of atherosclerosis, NIDDM and treated hypertension, body length and prostate cancer-related factors, such as stage, grade and PSA-level. This notion is in line with findings in the present report, that when patients with PSA-levels exceeding 50 ng/mL were included at the comparison between slow-growing and fast-growing BPH, the prevalence of NIDDM became statistically significant. Moreover, the prostate cancer stage became borderline-significant and the statistical significance level of the difference in prostate cancer grade and PSA level was increased. On the other hand, the difference between men with slow-growing and men with fast-growing BPH as regards systolic blood pressure, diastolic blood pressure and obesity, measured by body weight, BMI, waist measurement and hip measurement was decreased or disappeared. Finally, the difference between the slow-growing and the fast-growing group concerning fasting plasma insulin disappeared. Thus, the findings in the present study are in line with the assumption that men suffering from clinical prostate cancer with a PSA-level exceeding 50 ng/mL lose appetite and weight and, later on, develop a reduction in the fasting plasma insulin level and the blood pressure. The more stable factors, however, such as the prevalence of NIDDM, become statistically significant, as more patients are included in the comparison. Moreover, prostate cancer-related factors, such as prostate cancer stage and grade and PSA level, which proceed in the relentless cancer development, are not affected. It has not escaped our attention, assuming that there is a promoting effect of insulin on the prostate cancer growth, that the advancement of the prostate cancer growth in this way might bring about a decreased insulin level which reduces the further promotion of such growth.

It might be argued that in the present study the calculated BPH volume did not necessarily reflect the true BPH enlargement. The calculated transition zone volume was determined by subtracting 20 ml from the total prostate volume, as it was assumed that 20 ml was the prostate gland volume at the patient age of forty (14B). Consequently, the measured volume, in excess of 20 ml, would represent the BPH growth in the transition zone, as it has been reported that BPH selectively affects only the transition zone of the prostate gland (3B). We have recently reported measurements of both the total prostate gland volume and the transition zone volume in 114 men.

The validity coefficient between the total prostate gland volume and the transition zone volume was rs=0.97; p<0.0001, i.e. an almost perfect correspondence between the total prostate gland volume and the transition zone volume (18B). This means that the total prostate gland volume measured in the present report is a valid expression of the BPH volume.

To sum up, the results of the present report confirm the findings in previous reports in patients with recently diagnosed prostate cancer, that fast-growing BPH is a risk factor for NIDDM, hypertension, tallness, obesity, dyslipidaemia and hyperinsulinaemia. The study suggests that fast-growing BPH is a risk factor for developing clinical prostate cancer and, thus, supports the hypothesis of an association between the development of BPH and clinical prostate cancer. The study generates the hypotheses that clinical prostate cancer is a component of the metabolic syndrome and that insulin is a promoter of clinical prostate cancer development. In the present report, the hypothesis of an association between fast-growing BPH and the development of clinical prostate cancer has been tested by analysing grade, stage and PSA level crossectionally in men with recently diagnosed clinical prostate cancer with slow or fast-growing BPH. Another way of testing the same hypothesis would be to compare the prognosis of men with slow-growing and men with fast-growing BPH. Such a study is now in progress. If the above-mentioned hypotheses eventually are proved to be valid, it might be possible to develop effective preventive and therapeutic strategies, which might prevent or slow down the progression of clinical prostate cancer and reduce the need for surgery and hormonal treatment using methods available today.

TABLE 1B
The clinical, anthropometric, metabolic and insulin
profiles and prostate cancer stage, grade and PSA level
in men with clinical prostate cancer, PSA < 50 ng/mL,
and slow-growing or fast-growing benign prostatic
hyperplasia (dichotomies). Men with clinical prostate
cancer, PSA ≧ 50 ng/mL, were excluded.

| | BPH growth rate, mL/year | | |
|---|---|---|---|
| | <1.07 | ≧1.07 | |
| | n = 79 | n = 78 | P-value |
| Age, years | 73 | 72 | 0.490 |
| Prostate volume, mL | 39 | 75 | 0.001 |
| Atherosclerotic disease*, % | 24/77 = 31% | 20/78 = 26% | 0.440 |
| NIDDM**, % | 7/78 = 9% | 10/78 = 13% | 0.300 |
| Treated hypertension, % | 24/77 = 31% | 29/78 = 37% | 0.430 |
| Systolic blood pressure, mm Hg | 150 | 160 | 0.009 |
| Diastolic blood pressure, mm Hg | 85 | 90 | 0.020 |
| Tallness, cm | 174 | 177 | 0.001 |
| Body weight, kg | 79 | 84 | 0.001 |
| BMI*** | 26.0 | 27.7 | 0.005 |
| Waist measurement, cm | 96 | 101 | 0.001 |
| Hip measurement, cm | 101.5 | 103.0 | 0.003 |
| WHR**** | 0.96 | 0.97 | 0.150 |
| Triglycerides, mmol/l | 1.26 | 1.44 | 0.150 |
| HDL-cholesterol, mmol/l | 1.30 | 1.20 | 0.067 |
| Uric acid, umol/l | 339 | 339 | 0.46 |
| ALAT, ukat/l | 0.38 | 0.40 | 0.067 |
| Total cholesterol, mmol/l | 6.04 | 5.60 | 0.048 |
| LDL-cholesterol, mmol/l | 4.03 | 3.89 | 0.529 |
| Fasting plasma insulin, mU/l | 9.00 | 10.00 | 0.014 |
| T2***** | 26/79 = 33% | 19/78 = 24% | 0.160 |
| T3***** | 53/79 = 67% | 59/78 = 76% | 0.160 |
| G1****** | 36/79 = 46% | 23/78 = 29% | 0.029 |
| G2****** | 36/79 = 46% | 38/79 = 48% | |
| G3****** | 7/79 = 9% | 17/79 = 22% | |
| PSA, ng/mL******* | 13.0 | 17.3 | 0.016 |
| PSA-density | 0.36 | 0.23 | 0.001 |

*Atherosclerotic disease = Atherosclerotic disease manifestations
**NIDDM = Non-Insulin-Dependent Diabetes Mellitus
***BMI = Body Mass Index
****WHR = Waist/Hip Ratio
*****T = Clinical staging according to the 1992 TNM classification
******G = Differentiation rate:
G1 = High-differentiated cancer;
G2 = Moderately differentiated cancer;
G3 = Low-differentiated cancer
*******PSA = Prostate-Specific Antigen

TABLE 2B
The clinical, anthropometric, metabolic and insulin profiles
and prostate cancer stage, grade and PSA level in the
total material of men with clinical prostate cancer and
slow-growing or fast growing benign prostatic hyperplasia
(dichotomies). Men with clinical prostate cancer,
PSA ≧ 50 ng/mL, were included.

| | BPH growth rate, mL/year | | |
|---|---|---|---|
| | <1.15 | ≧1.15 | |
| | n = 110 | n = 110 | P-value |
| Age, years | 74 | 73 | 0.280 |
| Prostate volume, mL | 42.5 | 81.5 | 0.001 |
| Atherosclerotic disease*, % | 27/108 = 25% | 28/110 = 25% | 0.940 |
| NIDDM**, % | 8/109 = 7% | 18/110 = 16% | 0.039 |
| Treated hypertension, % | 39/108 = 36% | 35/109 = 32% | 0.530 |
| Systolic blood pressure, | 160 | 160 | 0.089 |
| mm Hg | | | |
| Diastolic blood pressure, | 85 | 90 | 0.040 |
| mm Hg | | | |
| Body length, cm | 174 | 177 | 0.036 |
| Body weight, kg | 79 | 82 | 0.024 |
| BMI*** | 26.1 | 26.4 | 0.220 |
| Waist measurement, cm | 97 | 98 | 0.035 |
| Hip measurement, cm | 102 | 103 | 0.110 |
| WHR**** | 0.96 | 0.97 | 0.23 |
| Triglycerides, mmol/l | 1.25 | 1.40 | 0.061 |
| HDL-cholesterol, mmol/l | 1.30 | 1.20 | 0.085 |
| Uric acid, umol/l | 336 | 341 | 0.930 |
| ALAT, ukat/l | 0.36 | 0.40 | 0.094 |
| Total cholesterol, mmol/l | 6.00 | 5.67 | 0.164 |
| LDL-cholesterol, mmol/l | 4.00 | 3.94 | 0.934 |
| Fasting plasma insulin, mU/l | 9.00 | 9.15 | 0.140 |
| T2***** | 27/110 = 25% | 18/110 = 16% | 0.09 |
| T3***** | 83/110 = 75% | 92/110 = 84% | 0.09 |
| G1****** | 45/109 = 41% | 27/110 = 25% | 0.002 |
| G2****** | 52/109 = 48% | 52/110 = 47% | |
| G3****** | 12/109 = 11% | 31/110 = 28% | |
| PSA, ng/mL******* | 18.6 | 30.7 | 0.001 |
| PSA-density | 0.44 | 0.36 | 0.52 |

*Atherosclerotic disease = Atherosclerotic disease manifestations
**NIDDM = Non-Insulin-Dependent Diabetes Mellitus
***BMI = Body Mass Index
****WHR = Waist/Hip Ratio
*****T = Clinical staging according to the 1992 TNM classification
******G = Differentiation rate:
G1 = Well differentiated cancer;
G2 = Moderately differentiated cancer;
G3 = Poorly differentiated cancer
*******PSA = Prostate-Specific Antigen

### REFERENCES

1B. Denis L, Griffiths K, Khoury S, Cockett A T K, McConnell, Chatelain C, Murphy G ar Yoshida I. In: 4^{th} International Consultation on Benign Prostatic Hyperplasia (BPH Proceedings 4. Plymebridge Distributors Ltd, Plymouth, United Kingdom, 1998.

2B. Kirby R, Christmas T and Brawer M. Chapter 3. In: Prostate Cancer, Mosby, an impri of Times Mirror International Publishers Limited, 1996.

3B. McNeal J. Pathology of benign prostatic hyperplasia Urol. Clin. N. Am. 1990; 17:47 86

4B. Breslow N, Chan C E, Dhom G, et al. Latent carcinoma of the prostate at autopsy seven areas. International Journal of Cancer 1977; 20:680-88

5B. Armenian H K, Lilenfeld A M, Diamond E L and Bross I D J. Relation between benig prostatic hyperplasia and cancer of the prostate. Lancet 1974; 2:115-17

6B. Tiblin G. Prostatic cancer: the men born in 1913 study. In: Medical Products Agenc workshop: pharmacological treatment of prostatic cancer, Vol 3. Uppsala, Swede Medical Products Agency. 1993:13-9

7B. Greenwald P, Kirmss V, Polan A K and Dick V S. Cancer of the prostate among m with benign prostatic hyperplasia. J. Natl. Cancer Inst. 1974; 53:335-40

8B. Hammarsten J, Andersson S, Holmén A, Högstedt B and Peeker R. Does transurethr resection of a clinically benign prostate gland increase the risk of developing clinic prostate cancer? A 10-year follow-up study. Cancer 1994; 74:2347-51

9B. Lund K, Ewertz M and Schou G. Breast cancer incidence subsequent to surgic reduction of the female breast. Scand. J. Plast. Reconstr. Surg. 1987; 21:209-12

10B. Gleason D F. Histologic grading of prostate cancer: a perspective. Human Patholo 1992; 23:273-9.

11B. Schröder F H, Hermanek P, Denis L, Fair W R, Gospodarowicz M K and Pavon Macaluso M. The TNM classification of prostate cancer. Prostate Suppl. 1992; 4:129-3

12B. Littrup P J, Williams C R, Egglin T K and Kane R A. Determination of prostate volun with transrectal US for cancer screening—Part II. Accuracy of in vitro and in vi techniques. Radiology 1991; 179:49-53.

13B. Terris M K and Stamey T A. Determination of prostate volume by transrectal ultrasoun J. Urol. 1991; 145:984-7.

14B Berry S J, Coffey D S, Walsh P C and Ewing L L. The development of human beni prostatic hyperplasia with age. J. Urol. 1984; 132:474-9.

15B. Gay J M. Clinical epidemiology and evidence-based medicine glossary. In: Terminolo Specific to Epidemiology, updated Aug. 22, 1999, page http://www.vetmed.wsu.edu./course-jmgay/GlossEpiTerminology.htm

16B. Hammarsten J, Högstedt B, Holthuis N and Mellström D. Components of the metabol syndrome—risk factors for the development of benign prostatic hyperplasia. Prosta cancer and prostatic diseases 1998; 1:157-62.

17B. Hammarsten J and Högstedt B. Clinical, anthropometric, metabolic and insulin profi of men with fast annual growth rates of benign prostatic hyperplasia Blood pressu 1999; 8:29-36.

18B. Hammarsten J and Högstedt B. Hyperinsulinaemia as a risk factor for developing beni prostatic hyperplasia European Urology 2001; 39:151-8.

19B. Reavan G M. Banting lecture 1988. Role of insulin resistance in human disease. Diabet 1988; 37:1595-1607.

20B. Rett K, Wicklmayr M and Mehnert H. New aspects of insulin resistance in hypertensio European Heart Journal 1994; 15 (Supplement C):78-81

21B. DeFronzo R A and Ferrannini E. Insulin resistance. A multifaceted syndrome responsible for NIDDM, obesity, hypertension, dyslipidemia, and atherosclerotic cardiovascular disease. Diabetes Care 1991;14:173-94

22B. Krotkiewski M. Role of muscle capillarization and morphology in the development of insulin resistance and metabolic syndrome. Presse Med. 1994; 23:1353-6

23B. Kalkhoff R K, Kim H J, Cerletty J and Ferrou C A. Metabolic effects of weight loss in obese subjects. Changes in plasma substrate levels, insulin and growth hormone responses. Diabetes 1971; 20:83-91

24B. Nilsson P M, Lindholm L H and Schersten B F. Life style changes improve insul resistance in hyperinsulinaemic subjects: a one-year intervention study of hypertensiv and normotensives in Dalby. J. Hypertens. 1992; 10:1071-78.

25B. Jimenez J, Zuniga-Guajardo S, Zinman B and Angel A. Effects of weight loss massive obesity on insulin and c-peptide dynamics: sequential changes in insul production, clearance, and sensitivity. Endocrinol Metab. 1987; 64:661-8

26B Morton M S, Blacklock N, Denis L and Griffiths K. Western diet and prostate cance does the available evidence justify dietary advice. In: Schröder F H. Recent advances prostate cancer and BPH. New York: The Parthenon Publishing Group Inc., 1996:61-7

27B Haber G B, Heaton K W, Murphy D and Burroughs L F. Depletion and disruption dietary fibre: effects on satiety, plasma-glucose, and serum-insulin. Lancet 1977; ii:67 82

28B. Karlström B, Vessby B, Asp N-G and Ytterfors A. Effects of four meals with differe kinds of dietary fibre on glucose metabolism in healthy subjects and non-insuli dependent diabetic patients. European Journal of Clinical Nutrition 1988; 42:519-526

29B. Björntorp P, De Jounge K, Sjöström L and Sullivan L. The effect of physical training c insulin production in obesity. Metabolism 1970; 19:631-38.

30B. Krotkiewski M, Bylund-Fallenius A-C, Holm J, Björntorp P, Grimby G ar Mandroukas K. Relationship between muscle morphology and metabolism in obe women: the effects of long-term physical training. Europ. J. Clin. Invest. 1983; 13:5-12

31B. DeFronzo R A, Sherwin R S and Kraemer N. Effect of physical training on insulin actic in obesity. Diabetes 1987; 36:1379-85

32B. Seals D R, Hagberg J M, Hurley B F, Ehsani A A and Holloszy J. O. Effects of enduran training on glucose tolerance and plasma lipid levels in older men and women. JAM 1984; 252:645-9.

33B. Eriksson K-F and Lindgärde F. Prevention of type 2 (non-insulin-dependent) diabet mellitus by diet and physical exercise. The 6-year Malmö feasibility study. Diabetolog 1991; 34:891-8.

34B. Hjermann I, Leren P, Norman N, Helgeland A and Holme I. Serum insulin response oral glucose load during a dietary intervention trail in healthy coronary high risk me the Oslo study. Scand. J. Clin. Lab. Invest. 1980; 40:89-94

35B. Landin K, Tengborn L and Smith U. Treating insulin resistance in hypertension wi metformin reduces both blood pressure and metabolic risk factors. J. Intern. Med. 199. 229:181-87

36B Pollare T, Lithell H and Berne C. A comparison of the effects of hydrochlorothiazi and captopril on glucose and lipid metabolism in patients with hypertension. N. Engl. Med. 1989; 321:868-73.

37B. Pollare T, Lithell H, Selinus I and Berne C. Application of prazosin is associated with a increase of insulin sensitivity in obese patients with hypertension. Diabetologia 198 31:415-20

38B. Swislocki A L M, Hoffman B B, Sheu W H-H, Chen Y-D I. and Reaven G M. Effect prozasin treatment on carbohydrate and lipoprotein metabolism in patients wi hypertension. Am. J. Med. 1989; 86 (Suppl.1B):14-8.

39B. Haenni A and Lithell H. Treatment with a beta-blocker with beta2-agonism improv glucose and lipid metabolism in essential hypertension. Metabolism 1994: 43:455-61.

40B. Andersson P-E, Johansson J, Berne C and Lithell H. Effects of selective alpha1 a beta1 adrenoreceptor blockade on lipoprotein and carbohydrate metabolism hypertensive subjects, with special emphasis on insulin sensitivity. J. Huma Hypertens. 1994; 8:219-26.

41B. Andersson P-E and Lithell H. Metabolic effects of doxazosin and enalapril i hypertriglyceridemic, hypertensive men. Relationship to changes in skeletal musc blood flow. Am. J. Hypertens. 1996; 9:323-33

42B. Shieh S-M, Sheu W H-H, Shen D-C, Fuh M M-T, Chen Y-D I. and Reaven G Glucose, insulin, and lipid metabolism in doxazosin-treated patients with hypertensio Am. J. Hypertens. 1992; 5:827-31.

Study 3: Clinical Prostate Cancer—a Component of the Metabolic Syndrome

Previous studies have suggested that benign prostatic hyperplasia (BPH) is a component of the metabolic syndrome (1C, 2C, 3C, 4C). This new concept has emerged from studies based on the clinical observation that diabetic and obese men seemed to have larger prostate glands than men without these conditions. Four reports have been published, suggesting that fast-growing BPH is a risk factor for the development of components of the metabolic syndrome, including non-insulin-dependent diabetes mellitus (NIDDM), hypertension, obesity, dyslipidaemia and hyperinsulinaemia (1C, 2C, 3C, 4C). Three of these reports were based on a patient material of 307 consecutive patients with lower urinary tract symptoms referred to our hospital (1C, 2C, 3C). The fourth study comprised 220 patients with recently diagnosed clinical prostate cancer (4C). The fact that the relationship between fast-growing BPH and components of the metabolic syndrome was found in two entirely different groups of patients supports the conclusion that BPH is a component of the metabolic syndrome. Moreover, this conclusion is supported by the recent report that a genetically hypertensive rat strain—spontaneously hypertensive rats—also develops BPH-like features (5C).

The metabolic syndrome is a multifaceted condition that occurs frequently in the general population. It is more common in men than in women. A great number of adults in industrialized countries, such as Sweden, develop the metabolic syndrome because of genetic, hormonal and/or lifestyle factors such as stress, smoking, certain nutritional excesses and physical inactivity (6C). This syndrome has been described as a single entity characterized by a defective insulin-mediated glucose uptake (7C, 8C, 9C). The primary metabolic abnormality of the metabolic syndrome is mainly localized to the muscle mass, the adipose tissue and the liver of the patients suffering from this syndrome, leading to insulin-resistance and secondary hyperinsulinaemia (10C).

In a previous study, it was found that there was an association between the development of BPH and the development of clinical prostate cancer (4C). The study generated the hypothesis that clinical prostate cancer also is a component of the metabolic syndrome. In the present study, this hypothesis was tested on 246 men with recently diagnosed clinical prostate cancer. If this hypothesis is true, patients with clinical prostate cancer of high stage and grade would have a larger prostate gland volume, a faster BPH growth rate and a more pronounced clinical, haemodynamic, anthropometric, metabolic and insulin profile than patients with clinical prostate cancer of a low stage and grade.

Patients and Methods

Two hundred and forty-six patients referred to the Urological Section, Department of Surgery, Varberg Hospital, Varberg, Sweden, in whom clinical prostate cancer was diagnosed, were consecutively included in this study. Clinical prostate cancer was defined as a prostate tumour indicated by a digital rectal examination or by ultrasound and verified histopathologically using the technique of transrectal ultrasound-guided automatic needle biopsy of the prostate gland. The core biopsy was morphologically classified as poorly differentiated (G3), moderately differentiated (G2) or well differentiated (G1) cancer by our histopathologists. The prostate cancer tumours were also subjected to clinical staging and classified in accordance with the 1992 TNM classification (11C). In an effort to classify the prostate cancer tumours into localized and advanced prostate cancer, the following criteria were used. Patients with clinical prostate cancer T2-3 and prostate-specific antigen (PSA)<50 were judged to have localized cancer, while those with prostate cancer T3 and PSA>50 were judged to have advanced prostate cancer. Most of the 220 patients were investigated because of symptoms generated by the prostate cancer tumours, but some had their prostate cancer discovered while seeking medical advice for other reasons. The median age of the 246 men was 73 years (range 49-91).

Varberg Hospital has a strictly defined catchment area with a permanent population of about 150,000 people. The Swedish health care system is organized in such a way that a great majority of men living in this area are referred to Varberg Hospital for medical care, including urologic problems.

Men with another malignant disease in their medical history, men with a significant body weight change (±10 kg) during the last 10 years, men on finasterid medication and men subjected to a previous transurethral resection of the prostate gland with unknown resection weight were excluded. Moreover, men subjected to hormonal manipulation (ablation of the testes, treatment with GnRH analogues, antiandrogens, testosterone, estrogens, steroids, insulin, thyroxin or growth hormone) were excluded. In this study, only the clinical cancer tumours, T2-T3, Nx, Mx were included because it was considered that inclusion of more advanced tumours could distort the evaluation of anthropometric, hormonal and metabolic data. It is obvious that men suffering from advanced clinical prostate cancer lose appetite and weight, which would affect several risk factors we wanted to assess in the present investigation. The biggest prostate cancer tumour included in the present study, as estimated by digital rectal examination and ultrasound, had a diameter of 2 cm which corresponds to a volume of 4.2 ml.

A patient was said to have had hypertension if this condition had been pharmacologically treated and NIDDM if this diagnosis was provided by the patient's medical records. Atherosclerotic disease manifestations include coronary artery disease, cerebrovascular disease and peripheral arterial insufficiency. Coronary artery disease includes symptoms of effort angina pectoris and a history of myocardial infarction. Cerebrovascular disease is defined as a history of stroke or a transitory ischemic attack (TIA) documented by the patient file. Patients with a history of arterial aneurysm, intermittent claudication, rest pain or peripheral gangrene caused by arterial insufficiency—whether subjected to surgery or not—were defined as patients with peripheral arterial insufficiency.

Data on blood pressure, waist and hip measure, tallness and body weight were collected. Moreover, the body mass index (BMI, kg per m²) and the waist/hip ratio (WHR) were calculated. The prostate gland was examined using digital rectal examination and ultrasound equipment (B&K Medical 3535). The prostate gland volume was determined by means of ultrasound using the ellipsoid method (12C, 13C). The age-adjusted prostate growth rate was calculated. In this calculation, the annual BPH growth rate was based on the assumption that the prostate growth rate is linear over time and that the prostate gland volume is 20 mL when the patient is 40 years old (14C). The following formula was used: total prostate gland volume—20 mL/age—40 years.

Blood samples were drawn from overnight-fasting patients. Serum to determine insulin was separated within one hour of sampling and stored at −20° C. until assayed. Fresh serum was analysed for total cholesterol, HDL-cholesterol, triglycerides, uric acid and alanine aminotransferase (ALAT, EC 2.6.1.2.).

Serum insulin was measured by means of a radioimmunoassay kit, Insulin RIA 100, from Pharmacia Diagnostics, Uppsala, Sweden, using a human insulin standard. Total cholesterol, HDL-cholesterol, triglycerides, uric acid and ALAT were analysed on a Synchrone CX7 instrument from Beckman Instruments Inc, Brea, Calif., USA, with reagents from the same supplier. HDL-cholesterol was measured in the supernatant after precipitation with dextran sulphate and magnesium chloride. LDL-cholesterol was calculated using the Friedewald formula. Total PSA was measured by means of Elecsys total PSA Immunoassay from Roche.

Since most variables in this report were not normally distributed, we preferred to use non-parametric statistics, i.e. the median value and the Mann-Whitney U-test for calculations of differences between groups, and the Chi-square or the Fisher test for calculations of differences in proportions between groups. A risk factor for developing clinical prostate cancer was defined as a factor that is statistically associated with the development of clinical prostate cancer.

The ethical aspects of this study were approved by the Ethical Committee of the Medical Faculty of the University of Göteborg, Göteborg, Sweden.

Results

Table 1C shows the prostate gland volume, the annual BPH growth rate and the clinical, haemodynamic, anthropometric, metabolic and insulin profiles in patients with clinical prostate cancer of stages T2 and T3, excluding men with clinical prostate cancer, PSA>50 ng/mL. Men with stage T3 had a bigger prostate gland volume, a faster BPH growth rate and were more obese, as determined by body weight, BMI, waist and hip measurement, than patients with T2 clinical prostate cancer.

Data on patients with stages T2 and T3 in the total material of patients with clinical prostate cancer, including patients with clinical prostate cancer, PSA>50 ng/ml, are given in Table 2C. At this comparison, subjects with a T3 tumour showed a statistical significance when it came to a higher prevalence of treated hypertension than men with 12 tumours.

Table 3C shows the prostate gland volume, the BPH growth rate and the haemodynamic, metabolic and insulin profiles in patients with grades 1C-3 clinical prostate cancer, excluding those with clinical prostate cancer, PSA>50 ng/ml. Patients with G1 tumours were compared with those who had G3 tumours. At this comparison, the differences between G1 and G3 clinical prostate cancer were as follows: Patients with a G3 tumour had a greater prostate gland volume, a faster BPH growth rate and were also more dyslipidaemic, as shown by a statistical significance with respect to a decreased HDL-cholesterol level and a borderline statistical significance with respect to an increased triglyceride level. Moreover, subjects with a G3 tumour showed a statistical significance as regards a higher plasma insulin level than those with a G1 tumour (FIG. 1B).

Table 4C gives the data on the total material of men with clinical prostate cancer, including patients with clinical prostate cancer, PSA>50 ng/ml, with regard to grades 1C-3C. Subjects with a G1 tumour were compared with those who had a G3 tumour. At this comparison, patients with a G3 tumour had a greater prostate gland volume and a faster BPH growth rate.

Discussion

The most important finding in the present report was that there was an association between advanced clinical prostate cancer, as measured by stage and grade, and several components of the metabolic syndrome, such as a greater prostate gland volume, a faster BPH growth rate, treated hypertension, obesity, dyslipidaemia and hyperinsulinaemia. This means that all these conditions are risk factors when it comes to the development of clinical prostate cancer. All these findings support the hypothesis that clinical prostate cancer is a facet of the metabolic syndrome, i.e. one of the diseases of western civilization in addition to NIDDM, hypertension, obesity, dyslipidaemia and hyperinsulinaemia These conditions are characterized by a defective insulin-stimulated glucose uptake and a secondary hyperinsulinaemia (10C). The findings suggest that the development of clinical prostate cancer also is associated with a defective insulin-stimulated glucose uptake and secondary hyperinsulinaemia, which generates a possibly promoting factor—hyperinsulinaemia.

This notion is supported by the findings in the present report that several factors that are known to be associated with hyperinsulinaemia, such as a great prostate gland volume, fast-growing BPH (1C, 2C, 3C, 4C), treated hypertension (8), obesity (9) and dyslipidaemia (9) were linked to more advanced clinical prostate cancer. Both a great prostate gland volume and fast-growing BPH were equally strong risk factors for developing clinical prostate cancer of high stage and grade. Treated hypertension and obesity were risk factors for developing high stage clinical prostate cancer, while dyslipidaemia and hyperinsulinaemia were risk factors for developing high grade clinical prostate cancer. In fact, a statistically significant relationship was found between the clinical prostate cancer grade and the fasting plasma insulin level. In patients with a well differentiated clinical prostate cancer, the median fasting plasma insulin level was 8.3 mU/l, while in those with moderately differentiated clinical prostate cancer it was 9.9 mU/l and in patients with poorly differentiated clinical prostate cancer 11.5 mU/l (FIG. 1B). A model of prostatic carcinogenesis has been proposed. It involves a morphological continuum from normal prostatic epithelial cells, through increasing grades of prostatic intra-epithelial neoplasia to early invasive carcinoma. Similarly, a model based on a series of putative genetic alterations involving tumour suppressor genes and oncogenes has been proposed (15C). It might be speculated that insulin is a promoter of these multistep transformations and, thus, a promoter of dedifferentiation. If this hypothesis is true, men with a high fasting plasma insulin value at baseline would have a poorer prognosis than men with a low fasting plasma insulin value. This hypothesis is currently being tested.

In our previous studies, it was consistently found that BPH, as measured by the annual BPH growth rate, was statistically significantly related to components of the metabolic syndrome, such as NIDDM, hypertension, tallness, obesity, dyslipidaemia and hyperinsulinaemia (1C-4C). In the present study, the relationship between clinical prostate cancer and the same components of the metabolic syndrome was not equally consistent. NIDDM and tallness were not statistically significantly related to clinical prostate cancer of high stage and grade. On the other hand, the prostate gland volume, the BPH growth rate, treated hypertension, obesity dyslipidaemia and hyperinsulinaemia showed highly significant correlations. Thus, it may be argued that clinical cancer is not as closely related to the metabolic syndrome as BPH. It must be recognized, however, that the stage and grade of the clinical prostate cancer, used as independent variables in the present report, are not as valid expressions of clinical prostate cancer as the annual BPH growth rate is of BPH. However, the fact that several independent manifestations of the metabolic syndrome, such as the prostate gland volume, the BPH-growth rate, hypertension, obesity, dyslipidaemia and hyperinsulinaemia go in the same direction strongly supports the conclusion that men with manifestations of the metabolic syndrome have a faster clinical prostate cancer development.

One finding which is difficult to explain in our reports concerning BPH and clinical prostate cancer is that atherosclerotic disease manifestations are not associated with neither BPH nor clinical prostate cancer (1C-4C), although several other components of the metabolic syndrome, such as NIDDM, treated hypertension, obesity, dyslipidaemia and hyperinsulinaemia showed highly significant correlations to BPH. Moreover, all these factors but NIDDM showed significant correlations to clinical prostate cancer. Furthermore, it is well recognized that all these conditions are risk factors for atherosclerotic disease manifestations (6C). It may be speculated that the reason for these unexpected observations in our reports might be that BPH and clinical prostate cancer peaks at 70 and 75 years of age, respectively. The mean time of death caused by atherosclerotic disease, however, takes place at a much lower age. Thus, our findings may imply, that there was a selective survival of individuals less liable to atherosclerotic disease manifestations in our patient materials. If this is true, it means that there is a survival selection bias when it comes to atherosclerotic disease manifestations in all our reports on BPH and clinical prostate cancer (1C-4C).

It has been claimed that clinical prostate cancer belongs to a group of diseases referred to as “Western diseases” because of their high prevalence in affluent Western countries and regions of Europe and North America, compared with Asian countries (19C). This group of diseases also includes atherosclerotic disease manifestations, NIDDM, hypertension, obesity and dyslipidaemia. The results of the present study support the concept that clinical prostate cancer belongs to this group of diseases. It has not escaped our notice that the results of the present report could explain the consistent finding of an increased age-adjusted incidence of clinical prostate cancer, when men originating in Asia or west Africa move to the United States (20C-21 C). The results of the present report suggest that the increased prostate cancer incidence in these men in the United States is due to life style factors, such as stress, smoking, certain nutritional excesses and physical inactivity. It is well recognized that these factors are related to a defective insulin-stimulated glucose uptake and a secondary hyperinsulinaemia (16C-18, 22C-37C) which, in turn, in accordance with our findings, is related to the development of clinical prostate cancer.

If the conclusions in the present report hold true, they mean, in the clinical setting, that any physician facing a patient with a great prostate gland volume, fast-growing BPH, hypertension, obesity, dyslipidaemia or/and hyperinsulinaemia, should consider the possibility that the patient has clinical prostate cancer. Another implication would be that more selective PSA-screening programmes could be considered, as high-risk groups of the populations could be identified. Still another implication would be that patients with a recently diagnosed localized clinical prostate cancer suffering from one or several of these risk factors could fare less well than patients without these risk factors and therefore would be in greater need of radical treatment. Moreover, the presence of one or several of these conditions in a patient with advanced clinical prostate cancer could mean a heavier tumour load and a poorer prognosis. Assuming that there is a promoting role of insulin in the development of clinical prostate cancer, it might be speculated that minor reductions of insulin levels might bring about a significantly lower growth rate of clinical prostate cancer. This would make primary, secondary and tertiary prevention and/or delaying measures possible. These minor reductions of the insulin level might be generated by factors such as weight reduction (16C-18C), dietary changes (22C-23C) or increased physical activity (24C-26C) or a combination of factors, such as increased physical activity and changed dietary regulation (17C, 28C) or modification of the diet and smoking habits (29C). Some drugs available today are also known to decrease the insulin levels and improve insulin sensitivity, such as metformin (30C), captopril (31C), prazosin (32C-33C), dilevalol (34C), doxazosin (35C-37C) and rosiglitazone (38C-39C).

In the present report, the results were analysed in a patient material divided into two different groups. In the first group, patients with clinical prostate cancer PSA>50 ng/mL, were excluded at the comparison between different stages and grades. In the second group, patients with clinical prostate cancer, PSA>50 ng/mL, were included at this comparison. The reason for this separation into two groups was that patients suffering from advanced clinical prostate cancer lose appetite and weight and, later on, develop a reduced fasting plasma insulin level, a reduction of several metabolic factors and of the blood pressure (16C, 17C, 18C). Consequently, these factors were included in the analysis when only those patients with clinical prostate cancer, PSA<50 ng/mL, were included at the comparison. Several other factors, such as the prevalence of NIDDM and treated hypertension, body tallness and prostate cancer-related factors, e.g. prostate cancer stage, grade and PSA level were found to be more stable and not affected by the relentless cancer development observed in a previous study (4C). Thus, these factors were analysed when patients with clinical prostate cancer, PSA>50 ng/mL, were included at the comparison. An interesting observation in the present study was that the prostate gland volume and the BPH growth rate seemed to belong to this group of stable factors, as the level of statistical significance increased or was unchanged when patients with advanced clinical prostate cancer, PSA>50 ng/mL, were included.

In conclusion, the results of the present report suggest that the prostate gland volume, the BPH growth rate, treated hypertension, obesity, dyslipidaemia and hyperinsulinaemia are risk factors for the development of clinical prostate cancer. Thus, our findings support the hypothesis that clinical prostate cancer is a component of the metabolic syndrome and that clinical prostate cancer patients may have the same metabolic abnormality of a defective insulin-mediated glucose uptake and secondary hyperinsulinaemia as patients with the metabolic syndrome. Thus, our data also support the hypothesis that hyperinsulinaemia is a promoter of clinical prostate cancer. In the present report, these hypotheses have been tested crossectionally by measuring the prostate gland volume, the BPH growth rate, the clinical, haemodynamic, anthropometric, metabolic and insulin profile in patients with recently diagnosed clinical prostate cancer of low stage and grade and comparing them with patients with clinical prostate cancer of high stage and grade. Another way of testing the same hypotheses would be to prospectively compare the prognosis of patients with recently diagnosed clinical prostate cancer and a low clinical, haemodynamic, anthropometric, metabolic and insulin profile with men having a high clinical, haemodynamic, anthropometric, metabolic and insulin profile. If these hypotheses are true, men with a low profile would have a more favourable prognosis than men with a high profile. Such a study is now in progress. If the above-mentioned hypotheses eventually are proved to be valid, it might be possible to develop effective preventive and therapeutic strategies, that might prevent or slow down the progression of clinical prostate cancer and reduce the need of surgery and hormonal treatment using methods available today.

TABLE 1C
The prostate gland volume, BPH-growth rate and the
haemodynamic, anthropometric, metabolic and insulin
profiles in patients with T2 and T3 clinical prostate
cancer, excluding men with prostate cancer
PSA* > 50 ng/ml.

| | Stage | | |
|---|---|---|---|
| | T2, PSA < | T3, PSA < | P-value |
| | 50 ng/ml | 50 ng/ml | T2 vs T3 |
| Age, years | 71.4 | 72.9 | 0.570 |
| n | 48 | 128 | |
| Prostate volume, ml | 44.5 | 56.0 | 0.001 |
| BPH growth rate, ml/year | 0.74 | 1.15 | 0.011 |
| Systolic blood pressure, mm Hg | 150 | 160 | 0.151 |
| Diastolic blood pressure, mm Hg | 85 | 90 | 0.159 |
| Body weight, kg | 79.0 | 81.0 | 0.056 |
| BMI** | 25.8 | 27.0 | 0.005 |
| Waist measurement, cm | 97.0 | 98.5 | 0.07 |
| Hip measurement, cm | 101.0 | 103.0 | 0.030 |
| WHR*** | 0.96 | 0.97 | 0.146 |
| Triglycerides, mmol/l | 1.46 | 1.33 | 0.737 |
| HDL-cholesterol, mmol/l | 1.30 | 1.20 | 0.203 |
| Uric acid, umol/l | 340 | 333 | 0.498 |
| ALAT, ukat/l | 0.37 | 0.39 | 0.749 |
| Total cholesterol, mmol/l | 6.1 | 5.8 | 0.431 |
| LDL-cholesterol, mmol/l | 4.0 | 3.9 | 0.836 |
| Fasting plasma insulin, mU/l | 8.7 | 10.0 | 0.101 |

*PSA = Prostate-Specific Antigen
**BMI = Body Mass Index
***WHR = Waist/Hip Ratio

TABLE 2C
The prostate gland volume, BPH growth rate and the
clinical, anthropometric profile and prostate cancer
grades in patients with T2 and T3 prostate cancer,
including patients with prostate cancer PSA* > 50 ng/ml.

Stage

| | | | P-value |
|---|---|---|---|
| | T2 | T3 | T2 vs T3 |
| Age, years | 71.4 | 72.9 | 0.017 |
| n | 48 | 198 | |
| Prostate volume, ml | 44.5 | 63.0 | 0.000 |
| BPH growth rate, ml/year | 0.74 | 1.26 | 0.000 |
| Atherosclerotic disease**, % | 28 | 26 | 0.470 |
| NIDDM***, % | 9 | 13 | 0.310 |
| Treated hypertension, % | 21 | 37 | 0.026 |
| Body tallness, cm | 176 | 175 | 0.490 |
| G1**** | 52% | 28% | 0.005 |
| G2**** | 38% | 48% | |
| G3**** | 10% | 24% | |
| PSA, ng/ml | 10.2 | 30.3 | 0.000 |
| PSA-density | 0.26 | 0.54 | 0.000 |

*PSA = Prostate-Specific Antigen
**Atherosclerotic disease = Atherosclerotic disease manifestations
***NIDDM = Non-Insulin-Dependent Diabetes Mellitus
**** G = Differentiation rate:
G1 = Well differentiated cancer;
G2 = Moderately differentiated cancer;
G3 = Poorly differentiated cancer

TABLE 3C
The prostate gland volume, BPH-growth rate and the
haemodynamic, anthropometric, metabolic and insulin
profiles in patients with Grades 1-3 clinical prostate
cancer. Patients with clinical prostate cancer,
PSA* > 50 ng/ml, were excluded.

| | Grade** | | | |
|---|---|---|---|---|
| | G1 | G2 | G3 | G1 vs. |
| | PSA < 50 | PSA < 50 | PSA < 50 | G3 |
| | ng/ml | ng/ml | ng/ml | P-value |
| Age, years | 72.5 | 72.0 | 73.0 | 0.990 |
| n | 68 | 78 | 30 | |
| Prostate volume, ml | 49.0 | 50.0 | 67.0 | 0.004 |
| BPH growth rate, ml/year | 1.01 | 1.04 | 1.43 | 0.005 |
| Systolic blood pressure, | 160 | 160 | 165 | 0.550 |
| mm Hg | | | | |
| Diastolic blood pressure, | 90 | 90 | 90 | 0.480 |
| mm Hg | | | | |
| Body weight, kg | 80.0 | 81.5 | 82.0 | 0.230 |
| BMI*** | 26.4 | 26.7 | 26.2 | 0.480 |
| Waist measurement, cm | 98.0 | 98.0 | 98.3 | 0.230 |
| Hip measurement, cm | 102.0 | 103.0 | 102.5 | 0.330 |
| WHR**** | 0.97 | 0.95 | 0.99 | 0.210 |
| Triglycerides, mmol/l | 1.27 | 1.30 | 1.53 | 0.065 |
| HDL-cholesterol, mmol/l | 1.26 | 1.30 | 1.10 | 0.019 |
| Uric acid, umol/l | 317 | 347 | 337 | 0.170 |
| ALAT, ukat/l | 0.36 | 0.40 | 0.38 | 0.440 |
| Total cholesterol, mmol/l | 6.0 | 5.7 | 6.1 | 0.730 |
| LDL-cholesterol, mmol/l | 4.0 | 3.8 | 4.1 | 0.850 |
| Fasting plasma insulin, | 8.3 | 9.9 | 11.5 | 0.019 |
| mU/l | | | | |

*PSA = Prostate-Specific Antigen
**G = Differentiation rate:
G1 = Well differentiated cancer;
G2 = Moderately differentiated cancer;
G3 = Poorly differentiated cancer
***BMI = Body Mass Index
****WHR = Waist/Hip Ratio

TABLE 4C
The prostate gland volume, BPH-growth rate and
the clinical and anthropometric profiles, prostate cancer
stage and PSA* level in patients with Grades 1-3
clinical prostate cancer. Patients with prostate cancer,
PSA* > 50 ng/ml, were included.

| | Grade** | | | |
|---|---|---|---|---|
| | | | | G1 vs. G3 |
| | G1 | G2 | G3 | P-value |
| Age, years | 73.0 | 73.0 | 73.0 | 0.820 |
| n | 81 | 112 | 52 | |
| Prostate volume, ml | 52.0 | 57.5 | 70.0 | 0.002 |
| BPH growth rate, ml/year | 1.08 | 1.18 | 1.37 | 0.003 |
| Atherosclerotic disease***, % | 35 | 21 | 25 | 0.100 |
| NIDDM****, % | 9 | 13 | 15 | 0.240 |
| Treated hypertension, % | 33 | 34 | 38 | 0.770 |
| Body tallness, cm | 1.75 | 1.75 | 1.75 | 0.940 |
| T2***** | 31% | 16% | 10% | 0.052 |
| T3***** | 69% | 84% | 90% | 0.052 |
| PSA*, ng/ml | 18.6 | 26.1 | 34.4 | 0.001 |
| PSA-density | 0.33 | 0.44 | 0.52 | 0.033 |

*PSA = Prostate-Specific Antigen
**G = Differentiation rate:
G1 = Well differentiated cancer;
G2 = Moderately differentiated cancer;
G3 = Poorly differentiated cancer
***Atherosclerotic disease = Atherosclerotic disease manifestations
****NIDDM = Non-Insulin-Dependent Diabetes Mellitus
*****T = Clinical staging according to the 1992 TNM classification

### REFERENCES

1C. Hammarsten J, Högstedt B, Holthuis N and Mellström D. Components of the metabolic syndrome—risk factors for the development of benign prostatic hyperplasia Prostate cancer and prostatic diseases 1998; 1:157-62.

2C. Hammarsten J and Högstedt B. Clinical, anthropometric, metabolic and insulin profile of men with fast annual growth rates of benign prostatic hyperplasia. Blood pressure 1999; 8:29-36

3C. Hammarsten J and Högstedt B. Hyperinsulinaemia as a risk factor for developing benign prostatic hyperplasia. Accepted for publication in European Urology

4C. Hammarsten J and Högstedt B. Fast-growing benign prostatic hyperplasia—a risk factor for developing clinical prostate cancer. Submitted for publication.

5C. Golomb E., Rosenzweig N., Eilam R. and Abramovici A. Spontaneous hyperplasia of the ventral lobe of the prostate in aging genetically hypertensive rats. J. Androl. 2000; 21:58-64

6C. Timar O., Sestier F and Levy E. Metabolic syndrome X: a review. Can J. Cardiol. 2000; 16(6):779-89.

7C. Reavan G M. Banting lecture 1988. Role of insulin resistance in human disease. Diabetes 1988; 37:1595-1607.

8C. Rett K, Wicklmayr M and Mehnert H. New aspects of insulin resistance in hypertension. European Heart Journal 1994; 15 (Supplement C):78-81

9C. DeFronzo R A and Ferrannini E. Insulin resistance. A multifaceted syndrome responsible for NIDDM, obesity, hypertension, dyslipidemia, and atherosclerotic cardiovascular disease. Diabetes Care 1991;14:173-94

10C. Krotldewski M. Role of muscle capillarization and morphology in the development of insulin resistance and metabolic syndrome. Presse Med. 1994; 23:1353-6

11C. Schröder F H, Hermanek P, Denis L, Fair W R, Gospodarowicz M K and Pavone-Macaluso M. The TNM classification of prostate cancer. Prostate Suppl. 1992; 4:129-38

12C. Littrup P J, Williams C R, Egglin T K and Kane R A. Determination of prostate volume with transrectal US for cancer screening—Part II. Accuracy of in vitro and in vivo techniques. Radiology 1991; 179:49-53.

13C. Terris M K and Stamey T A. Determination of prostate volume by transrectal ultrasound. J. Urol. 1991; 145:984-7.

14C. Berry S J, Coffey D S, Walsh P C and Ewing L L. The development of human benign prostatic hyperplasia with age. J.Urol. 1984; 132:474-9.

15C. Captcoat M J, Chapter 1: Natural History. In: Captcoat M J. The management of advanced prostate cancer. Oxford: Blackwell Science Ltd., 1996:8-9

16C. Kalkhoff R K, Kim H J, Cerletty J and Ferrou C A. Metabolic effects of weight loss in obese subjects. Changes in plasma substrate levels, insulin and growth hormone responses. Diabetes 1971; 20:83-91

17C. Nilsson P M, Lindholm L H and Scherstén B F. Life style changes improve insulin resistance in hyperinsulinaemic subjects: a one-year intervention study of hypertensives and normotensives in Dalby. J. Hypertens. 1992; 10:1071-78.

18C. Jimenez J, Zuniga-Guajardo S, Zinman B and Angel A. Effects of weight loss in massive obesity on insulin and c-peptide dynamics: sequential changes in insulin production, clearance, and sensitivity. Endocrinol Metab. 1987; 64:661-8

19C. Morton M S, Blacklock N, Denis L and Griffiths K Western diet and prostate cancer: does the available evidence justify dietary advice. In: Schröder F H. Recent advances in prostate cancer and BPH. New York: The Parthenon Publishing Group Inc., 1996:61-74

20C. Muir CS, Nectoux J and Staszewski J. The epidemiology of prostatic cancer: Geographical distribution and time trends. Acta Oncol. 1991; 30:133-40.

21C. Shimizu H, Ross R K, Bernstein L, Yatani R, Henderson B E and Mack T M. Cancers of the prostate and breast among Japanese and white immigrants in Los Angeles County. Br. J. Cancer 1991; 63:963-66.

22C. Haber G B, Heaton K W, Murphy D and Burroughs L F. Depletion and disruption of dietary fibre: effects on satiety, plasma-glucose, and serum-insulin. Lancet 1977; ii:679-82

23C. Karlström B, Vessby B, Asp N-G and Ytterfors A. Effects of four meals with different kinds of dietary fibre on glucose metabolism in healthy subjects and non-insulin-dependent diabetic patients. European Journal of Clinical Nutrition 1988; 42:519-526

24C. Björntorp P, De Jounge K, Sjöström L and Sullivan L. The effect of physical training on insulin production in obesity. Metabolism 1970; 19:631-38.

25C. Krotkiewski M, Bylund-Fallenius A-C, Holm J, Björntorp P, Grimby G and Mandroukas K. Relationship between muscle morphology and metabolism in obese women: the effects of long-term physical training. Europ. J. Clin. Invest. 1983; 13:5-12

26C. DeFronzo R A, Sherwin R S and Kraemer N. Effect of physical training on insulin action in obesity. Diabetes 1987; 36:1379-85

27C. Seals D R, Hagberg J M, Hurley B F, Ehsani A A and Holloszy J. O. Effects of endurance training on glucose tolerance and plasma lipid levels in older men and women. JAMA. 1984; 252:645-9.

28C. Eriksson K-F and Lindgärde F. Prevention of type 2 (non-insulin-dependent) diabetes mellitus by diet and physical exercise. The 6-year Malmö feasibility study. Diabetologia 1991; 34:891-8.

29C. Hjermann I, Leren P, Norman N, Helgeland A and Holme 1. Serum insulin response to oral glucose load during a dietary intervention trial in healthy coronary high risk men: the Oslo study. Scand. J. Clin. Lab. Invest. 1980; 40:89-94

30C. Landin K, Tengborn L and Smith U. Treating insulin resistance in hypertension with metformin reduces both blood pressure and metabolic risk factors. J. Intern. Med. 1991; 229:181-87

31C. Pollare T, Lithell H and Berne C. A comparison of the effects of hydrochlorothiazide and captopril on glucose and lipid metabolism in patients with hypertension. N. Engl. J. Med. 1989; 321:868-73.

32C. Pollare T, Lithell H, Selinus I and Berne C. Application of prazosin is associated with an increase of insulin sensitivity in obese patients with hypertension. Diabetologia 1988; 31:415-20

33C. Swislocki A L M, Hoffman B B, Sheu W H-H, Chen Y-D I. and Reaven G M. Effect of prozasin treatment on carbohydrate and lipoprotein metabolism in patients with hypertension. Am. J. Med. 1989; 86 (Suppl.1B):14-8.

34C. Haenni A and Lithell H. Treatment with a beta-blocker with beta2-agonism improves glucose and lipid metabolism in essential hypertension. Metabolism 1994: 43:455-61.

35C. Andersson P-E, Johansson J, Berne C and Lithell H. Effects of selective alpha1 and beta1 adrenoreceptor blockade on lipoprotein and carbohydrate metabolism in hypertensive subjects, with special emphasis on insulin sensitivity. J. Human. Hypertens. 1994; 8:219-26.

36C. Andersson P-E and Lithell H. Metabolic effects of doxazosin and enalapril in hypertriglyceridemic, hypertensive men. Relationship to changes in skeletal muscle blood flow. Am. J. Hypertens. 1996; 9:323-33

37C. Shieh S-M, Sheu W H-H, Shen D-C, Fuh M M-T, Chen Y-D I. and Reaven G M. Glucose, insulin, and lipid metabolism in doxazosin-treated patients with hypertension. Am. J. Hypertens. 1992; 5:827-31.

38C. Whitcomb R W and Saltiel A R. Expert Opin. Inves. Drug. 1995; 4(12):1299-1309

39C. Elbrecht A. Chen Y, Cullinan C A et al. Biochem Biophys Res Commun. 1996; 224:431-37

1. A method for delaying or preventing an increase in prostate gland volume in humans or non-human mammals, which method comprises the administration of an effective, non-toxic and pharmaceutically acceptable amount of an insulin sensitiser or a pharmaceutically acceptable derivative thereof.

2. A method according to claim 1, for delaying or preventing an increase in total prostate gland volume.

3. A method according to claim 1 or claim 2, wherein the increase in total prostate gland volume is associated with the onset and/or development of benign prostatic hyperplasia (BPI).

4. A method according to any one of claims 1 to 3, for delaying or preventing an increase in volume of the transition zone (TZ) of the prostate gland.

5. A method according to any one of claims 1 to 4, for the delay or prevention of prostate volume increase in fast growing BPH.

6. A method according to claim 1, wherein the delay or prevention of increase in prostate gland volume results in a delay or prevention of onset of clinical prostate cancer, particularly in those having fast growing BPH.

7. A method according to claim 6, wherein the BPH is fast growing BPH.

8. A method for the treatment and/or prophylaxis of benign prostatic hyperplasia, in humans or non-human mammals, which method comprises the administration of an effective, non-toxic and pharmaceutially acceptable amount of an insulin sensitiser or a pharmaceutically acceptable derivative thereof.

9. A method according to any one of claims 1 to 8, wherein the insulin sensitiser is a thiazolidinedione.

10. A method according to any one of claims 1 to 8, wherein the insulin sensitiser is 5-(4-[2-(N-methyl-N-(2-pyridyl)amino)ethoxy]benzyl)-2,4-thiazolidinedione (Compound (I)), or a pharmaceutically acceptable derivative thereof.

11. A method according to any one of claims 1 to 8, wherein the insulin sensitiser is 5-[4-[2-(5-ethylpyridin-2-yl)ethoxy]benzyl]thiazolidine-2,4-dione (or pioglitazone), or a pharmaceutically acceptable derivative thereof.

12. A method according to any one of claims 1 to 8, wherein the insulin sensitiser is 2(S)-(2-benzoyl-phenylamino)-3-{4-[2-5-methyl-2-phenyl-oxazol-4-yl)-ethoxy]-phenyl}-propionic acid or a pharmaceutically acceptable derivative thereof.
